(19) United States
(12) Patent Application Publication
(10) Pub. No.: US 2003/0008024 A1
(43) Pub. Date: Jan. 9, 2003

(54) Therapeutic composition of herbal extracts and the uses thereof

(75) Inventor: Keyong Ren, Denville, NJ (US)

Correspondence Address: Yunling Ren, Esq., Cohen, Pontani, Lieberman & Pavane, 551 Fifth Avenue, Suite 1210, New York, NY, 10176, US

(73) Assignee: Advanced Herbal Therapeutics, Inc.

(21) Appl. No.: 09/989,568

(22) Filed: Nov. 20, 2001

Related U.S. Application Data
(60) Provisional application No. 60/273,422, filed on Mar. 5, 2001.

(51) Int. Cl. A61K 35/78

(52) U.S. Cl. 424/759; 424/773

(57) ABSTRACT

A novel therapeutic composition comprises three novel herbal extracts: Herba epimedium Extract, Rhizoma Drynaria fortunei (kze.) J. Sm Extract and Radix Tripterygium hypoglaucum (L'evl.) Hutch Extract, which are prepared through standardized manufacturing processes, respectively. A new formulation AHT-323 contains these three herbal extracts. The novel therapeutic composition described in the present application can be used for treating autoimmune diseases such as rheumatoid arthritis, inflammatory disorders and pain.

### RELATED APPLICATIONS

This application claims priority from U.S. Provisional Patent Application Serial No. 60/273,422 which was filed on Mar. 05, 2001. The contents of the provisional application are hereby incorporated by reference in its entirety.

### BACKGROUND OF THE INVENTION

1. Field of the Invention

The present invention relates to a novel composition of herbal extracts and its therapeutic uses. In particular, the invention is directed to a pharmaceutical composition containing Tripterygium hypoglaucum (L'evl.) Hutch extract, Drynaria fortunei (Kze.) J. Sm. Extract and Herba epimedium extract for treating autoimmune and inflammatory diseases.

2. Description of the Related Art

Arthritis is the leading chronic condition that causes American workers to lose more time than any other affliction. More than 50 million Americans are affected. It costs the U.S. economy $64.8 billion annually in medical care and lost wages.

Rheumatoid arthritis (RA) is a form of arthritis in which inflammation occurs primarily in the joint membranes or tissues (synovitis). Other tissues such as heart, lungs, eyes, nerves and muscles can also be affected in some cases. The disease is generally caused by autoimmune disorders. The typical symptoms of joint inflammation include swelling, pain and stiffness, especially in the morning, in specific joints on both sides of the body. In systemic cases, it can cause symptoms throughout the body, including tiredness, decreased appetite, weight loss, and mild fever.

For years, drug therapy has played a key role in symptom relieves and disease management of rheumatoid arthritis. Today, non-steroidal anti-inflammatory drugs (NSAID), such as aspirin, are the most commonly used anti-arthritis agents. They are quite effective in the treatment of inflammation and pain. While aspirin and other conventional NSAIDs can cause serious side effects when taken regularly and at high dosages, Cyclooxygenase 2 (COX-2) inhibitor, a new class of NSAID, has demonstrated significant efficacy with no obvious side effects. Yet NSAID drugs cannot inhibit the progression of RA.

Corticosteroids, the synthetic versions of the body's cortisone hormone, are used to inhibit RA progression. They effectively reduce inflammation and suppress auto-immunity. The most commonly prescribed corticosteroids are prednisone and dexamthasone. Long-term use, however, can produce such side effects as weight gain, rounding of the face, thinning of the skin and bone, acne, easy bruising, high blood pressure, and an increased risk of diabetes, infection and stomach ulcers. As a result, they are generally used only for short periods of time during acute flare-ups.

Anti-malaria drugs, such as chloroquine and hydroxychloroquine, and anti-cancer drugs including methotrexate, azathioprine and cyclosphosphamide are also used for the disease management. Both act to suppress inflammatory reactions and immune responses. Yet various drugs in this category can cause diarrhea, rashes, anemia, low white blood cell count, and increased risk of infection. Methotrexate can cause serious liver and lung problems. Some anti-malarial drugs can affect the eyes. In addition, it can take months before these drugs produce any beneficial effect. Nevertheless, the costs of those drugs are significant. There exists a need for development of more safe and effective medications to control the symptoms and progression of the disease at lower price.

Herbal medicines have emerged as a unique approach for meeting the need for safe, effective and relatively inexpensive new remedies for a variety of disorders. According to the World Health Organization (WHO), 4 billion people, i.e., about 80% of the world population, use herbal medicines for some aspects of primary health care. In the US, a recent study on trends in alternative medicine use indicated a 47.3% increase from 1990 to 1997 with 629 million total visits to alternative medicine practitioners exceeding 386 million total visits to all US primary care physicians in 1997. Herbal medicines represent the fastest growing segment among all of alternative medicine. The herbal medicines are produced in different forms, which range from crude, decocted herbs to refined, concentrated and standardized extracts. The health benefit from taking those herbals also varies with the quality of the products and the knowledge of consumers on the products. Some of the products have to be used under a physician's supervision, particularly those indicated for serious diseases although the majority of herbal medicines are generally safe. It is essential to conduct well-controlled clinical studies under guidance of regulatory authorities for herbal therapeutics to validate their safety and efficacy.

It has been known that Tripterygium hypoglaucum (L'evl.) Hutch, Drynaria fortunei (Kze.) J. Sm.and Herba epimedium contain certain substances that are effective for treating autoimmune and inflammatory diseases. Herba pimedium and Drynaria fortunei (Kze.) J. Sm. were documented in the first edition (1955) of the “Pharmacopoeia of the People's Republic of China”. and Tripterygium hypoglaucum (L'evl.) Hutch was approved by Chinese regulatory agency as a drug for arthritis in 1970s. All three herbal plants are also documented in the most recent edition (1995) of the “Pharmacopoeia of the People's Republic of China”.

However, the active ingredients in these plants have never been identified and isolated from these plants. Traditionally, these plants are used for treating diseases with minimum preparation, i.e., they are extracted simply with boiling water. Such water extracts contain very low percentage of the active ingredients, if at all, in liquid forms. Patients must take large quantities of such water extracts to obtain minimum treatment amounts of the active ingredients. In reality, the optimum treatment doses of each active ingredient in each of these plant can hardly be achieved due to the limited amounts of the liquids that a patient can possibly consume. As a result, efficacy and potency of the drug are inevitably compromised. Another drawback of such traditional use of these herbal medicines are the lack of quantification of the active ingredients so that it can be difficult to monitor such treatment and the result is unpredictable.

AHT-323 Capsule is a novel pharmaceutical form of the traditional Chinese medicines Tripterygium hypoglaucum (L'evl.) Hutch, Drynaria fortunei (Kze.) J. Sm.and Herba epimedium, which contains highly concentrated active ingredients extracted and isolated from these herbal plants. It is targeted for the treatment of rheumatoid arthritis. Unlike the above describe traditional use of these herbal plants, where the active ingredients of these plants have never been isolated and quantified, AHT-323 is made following a standardized, quantified and reproducible pharmaceutical process. AHT-323 contains three active botanical ingredients, Herba pimedium extract, Drynaria fortunei (Kze.) J. Sm. extract, and Tripterygium hypoglaucum (L'evl.) Hutch extract.

AHT-323 is currently manufactured in China. The manufacturing process is standardized and quality controlled. Analytical techniques, such as high performance liquid chromatography (HPLC) and thin layer chromatography (TCL) were employed for quality control. Major fractions and/or active principles from the active ingredients were selected as the “fingerprints”, which include icariin from Herba pimedium, naringin from Drynaria fortunei (Kze.) J. Sm. and triptolide from Tripterygium hypoglaucum (L'evl.) Hutch. The chemical properties of these fractions have been identified and their quantities and/or qualities are determined to ensure batch to batch consistency. Commercial standards are used when appropriate.

Advanced pharmacological studies using western methodology have demonstrated that AHT-323 acts to suppress both inflammation and autoimmunity and effectively treats a number of experimental animal models of human RA, including rat and mouse models of adjuvant arthritis, and mouse model of delayed type hypersensitivity induced by DNFB. AHT-323 was also shown to significantly inhibit the activities of IL-1, IL-2, IL-6, and TNF, as well as macrophage, lymphocyte and NK cell.

Acute and chronic toxicology studies in animals provided strong safety support to use the drug in RA patients. The acute toxicity studies were conducted in both mice and rats. The LD₅₀ of AHT-323 is determined to be 2.86±0.32 g/kg B. W. in mice and 4.10±0.45 g/kg B. W. in rats, which are approximately 143 and 205 times of the highest clinical dosages used, respectively. Chronic toxicological studies of AHT-323, conducted in both rats and dogs, demonstrate that AHT-323 is safe to use and shows no significant toxic effects at 0.3 g/kg in rats and 0.08 g/kg in dogs which are 15 or 4 times of the clinical dosage. Although AHT-323 at those doses inhibit formation of sperm, the effect is reversible and it will return to normal upon two month of drug withdrawal.

Pilot clinical tests conducted on patients with RA have indicated its significant efficacy in relieving the symptoms of inflammation and pain with minimal toxicity. The efficacy was also found significant in the patients who failed to respond to conventional chemical drug therapy. Patients were well tolerated at the effective dosage used and no serious adverse event was identified. The reported subjective and objective benefits of AHT-323 on patients with RA warrant further testing of the drug in an expanded size of patient population.

### SUMMARY OF THE INVENTION

An object of the present invention is to encompass a novel composition using a novel extracts from the traditional herbal medicine Tripterygium hypoglaucum (L'evl.) Hutch, Herba epimedium, and Drynaria fortunei (Kze.) J. Sm. These three novel herbal extracts are prepared by standardized processes, so that the major active ingredient in each of these extracts falls within a specified range. Specifically, the triptolide content in tripterygium hypoglaucum (L'evl.) Hutch extract is about 0.035% to about 0.045% by weight; the naringin in Drynaria fortunei (Kze.) J. Sm. is about 40% to about 50% by weight; the icariin content in Herba epimedium extract is about 10% to about 20% by weight.

A particular novel therapeutic composition encompassed by the present invention is AHT-323, as hereinafter defined.

Another object of the present invention is to develop standardized and repeatable manufacturing processes for preparing these three novel herbal extracts that meet the above mentioned active ingredient specifications.

A further object of the present invention is to develop methods of treating patients having autoimmune diseases such as rheumatoid arthritis, inflammatory disorders and pain by administering the novel therapeutic composition encompassed by the present invention. More specifically, a patient who is subject to such treatment is given about 60 to about 360 mg of the Herba epimedium Extract, about 25 to about 150 mg of the Rhizoma Drynaria fortunei (kze.) J. Sm Extract and about 25 to about 150 mg of the Radix Tripterygium hypoglaucum (L'evl.) Hutch Extract daily.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings wherein like references denote similar characters:

FIG. 1. Determination of triptolide (C₂₀H₂₄O₆) content of Radix Tripterygium hypoglaucum (L'evl.) Hutch extract, one of the three active ingredients of drug substance of AHT-323, by HPLC. The herbal extract was manufactured using a combination of extraction technologies including water extraction and big-pore polymeric absorbent column chromatography as described in the text. Sample for HPLC analysis was prepared as described in the text. HPLC was performed by using a Discovery C18 (250×4.6 mm) column, with a mobile phase of acetonitrile-0.2% phosphoric acid (30:70), a flow rate of 1.0 ml/min and a UV monitor set at 218 nm. The arrow indicates triptolide, the concentration of which is determined to be 0.045%.

FIG. 2. Determination of naringin (C₂₇H₃₂O₁₄.2H₂O) content of Rhizoma Drynariae extract, one of the three active ingredients of drug substance of AHT-323, by HPLC. The herbal extract was manufactured using a combination of extraction technologies including 95% alcohol (w/v) extraction and column chromatography with D101 big-pore polymeric absorbent as described in the text. Sample for HPLC analysis was prepared as described in the text. HPLC was performed by using a octadecylsilane bonded silica gel as the stationary phase and methanol-acid acetic-water (35:4:61) as the mobile phase. The wavelength of the UV detector is 283 nm. The arrow indicates naringin, the concentration of which is determined to be 46.7% on the dried basis.

FIG. 3. Determination of icariin (C₃₃H₄₀O15) content of Herba epimedii extract, one of the three active ingredients of drug substance of AHT-323, by HPLC. The herbal extract was manufactured using a combination of extraction technologies including water extraction and polyamide column chromatography as described in the text. Sample for HPLC analysis was prepared as described in the text. HPLC was performed by using a 18-alkyl silane bonded silica gel as the stationary phase and acetonitrile-water (30:70) as the mobile phase; The wavelength of the detector is 270 nm. The arrow indicates icariin, the concentration of which is determined to be 13%.

FIG. 4. Prophylactic effects of AHT-323 on primary injury of adjuvant arthritis rats.

FIG. 5. Prophylactic effects of AHT-323 on secondary injury of adjuvant arthritis rats.

FIG. 6. Therapeutic effects of AHT-323 on primary injury of adjuvant arthritis rats.

FIG. 7. Therapeutic effects of AHT-323 on secondary injury of adjuvant arthritis rats.

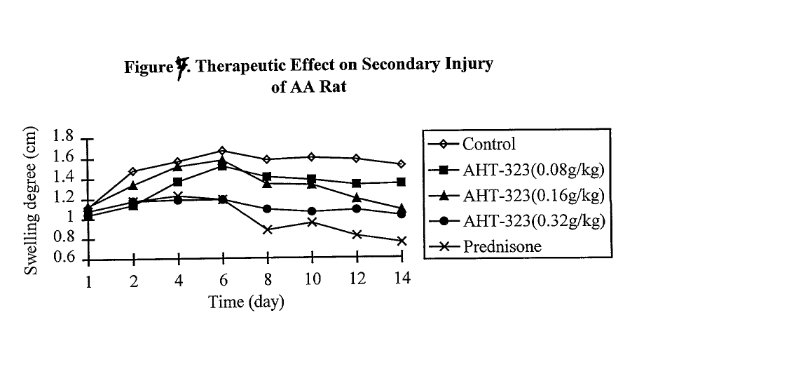

### DETAILED DESCRIPTION OF THE PRESENTLY PREFERRED EMBODIMENTS

AHT-323 is a multi-herb botanical product that comprises active botanical extracts from three different herbals native to China. The three herbals include: Tripterygium hypoglaucum (L'evl.) Hutch, Herba epimedium, and Drynaria fortunei (Kze.) J. Sm. Preferably, each of these three herbal extracts is prepared as described in the following examples, which has never been used prior to the present invention.

### EXAMPLE 1

### A Method for Preparation of Tripteryium hypoglaucum (L'evl.) Hutch Extract.

Raw Material:

Tripterygium hypoglaucum (L'evl.) Hutch belongs to family Celastraceae. It grows in bush facing the sun or loose woods mainly in Yunnan, Guangxi, and Sichuan provinces of China. The plant is harvested during fall season, and its roots are cleaned, peered from skin, dried, and used for the preparation of the herbal extract.

Extraction Process:

a. Water extraction

Cut raw material of dried roots into pieces and soak in water for 12 hours. Extract the raw herb with boiling water for 2 hours. Filtrate the extract through a filter, collect the passing through supernatant. Repeat extraction of the remaining of raw herbs with boiling water for 2 hours each and filtration, twice. Combine the three supernatants and discard the remaining of raw herbs.

b. Column chromatography extraction

Apply the supernatant from water extraction the column packed with big-pore polymeric absorbent, and then wash with water thoroughly. Elute the column with 80% ethanol while measuring the elution with an ethanol meter. Collect the elution when ethanol is detectable.

c. Solvent recovery

Recover the ethanol solvent from the solution above for reuse with reduced pressure while have the elution concentrated.

d. Vacuumed lyophilize

Have the condensed elution vacuum-lyophilized to dry. Have the powder so made passing through 80-mess particle sieve. Collect the 100% passing through powder for testing and encapsulation.

Method for Testing of Triptolide Content

a. Sample Preparation:

Six grams of the Tripterygium hypoglaucam extract are weighed up accurately and dissolved in small volume of 95% alcohol while mixing with solid neutro-alumina (about 1:5 w/w). The mixture is transferred into a Soxhlet extractor after complete evaporation of alcohol, and then extracted using petroleum ether via reflux extraction for 3 hours. Use chloroform to reflux extract for 6 hours after complete evaporation of petroleum ether. Retrieve chloroform, and dissolved the extract in methanol solution up to 10 ml in a 10 ml volumetric bottle. The preparation is used as the test sample solution, which is filtered with super-filtration membrane prior to injection onto HPLC.

b. Content Determination: HPLC Method

Mobile phase: acetonitrile: 0.2% phosphoric acid=30:70

Chromatographic column: Discovery C18 (250×4.6 mm)

Flow rate: 1.0 ml/min

Wave length: 218 nm

Extract Specification

Tripterygii hypoglaucum (L'evl.) Hutch extracts is standardized and quality controlled to contain a range of 0.035% to 0.045% (w/w) triptolide (C₂₀H₂₄O₆).

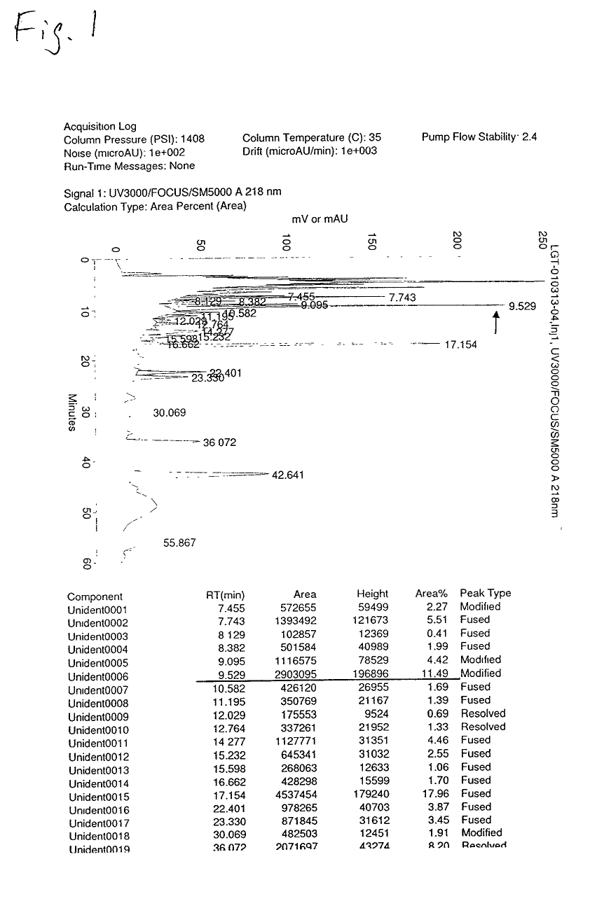

The result of HPLC determination of triptolide content is shown in FIG. 1.

### EXAMPLE 2

### A Method for Preparation of Drynaria fortunei (Kze.) J. Sm. Extract.

Raw Material:

Drynaria fortunei (Kunze) J. Sm belongs to family Polypodiaceae. The plant is perennial epiphytic herbage and grows on tree stems or rocks, mainly in Hubei, Zhejiang provinces and southwest of China. The rhizome is collected all year round, cleaned, dried and used for the extract preparation.

Extraction Process:

Cut raw herb into pieces and prepare coarse herbal powder using grinder. Put the coarse powder into extractor, add 95% alcohol at a volume 5 times the weight of herb, soak for 16 hours at 60° C. Filter and collect the filtrate. Soak the herb sediment again with 4 times volume of 95% alcohol (w/v) at 60° C. for 22 hours filtrate. Combine the two filtrate solutions.

Concentrate the filtrate solution via reduced pressure and have alcohol recovered for reuse. Add hot water (1/10 of the volume prior to concentration process above), left overnight. Eliminate insoluble material, add 2-time volume of water to dilute the solution, filter, and then load the filtrate solution onto a D101 big-pore polymeric absorbent column. Wash the column with 2-column volume water, and 4-column volume of 20% alcohol, respectively. Elute the column with 2-column volume of 80% alcohol, and collect the 80% alcohol elution.

Concentrate the 80% alcohol elution with reduced pressure and have alcohol recovered, and obtain condensed extract. Have part of solvent volatilized in 100° C. water bath, and dried in vacuum at 80° C. Obtain brownish yellow powder using grinder and weight the powder.

Extract Specification:

Rhizoma Drynariae extract is standardized and quality controlled to contain naringin (C₂₇H₃₂O₁₄.2H₂O) content in a range of 40-50% on the dried basis by a methodology of HPLC.

Methodology of HPLC:

Chromatographic system and system suitability Use octadecylsilane bonded silica gel as the stationary phase and methanol-acid acetic-water (35:4:61) as the mobile phase. The wavelength of the detector is 283 nm. The number of theoretical Preparation of reference solution Weigh accurately 20 mg of naringin CRS, dried to constant weight at 110° C., in a 100 ml volumetric flask, dilute with methanol to volume, and mix well. Accurately measure 3 ml in a 10 ml volumetric flask, dilute with methanol to volume, and mix well (containing 60 μg of narigin per ml).

Preparation of test solution Weigh accurately 0.25 g of the extract powder, add 30 ml of methanol, heat under reflux on a water bath for 3 hours, cool, and filter. Transfer the filtrate to a 50 ml volumetric flask, wash the container with a small quantity of methanol for several times, filter the washings to the same flask, dilute with methanol to volume, and mix well.

Procedure Inject accurately 10 μl of each of the reference solution and the test solution into the column, determine and calculated the content.

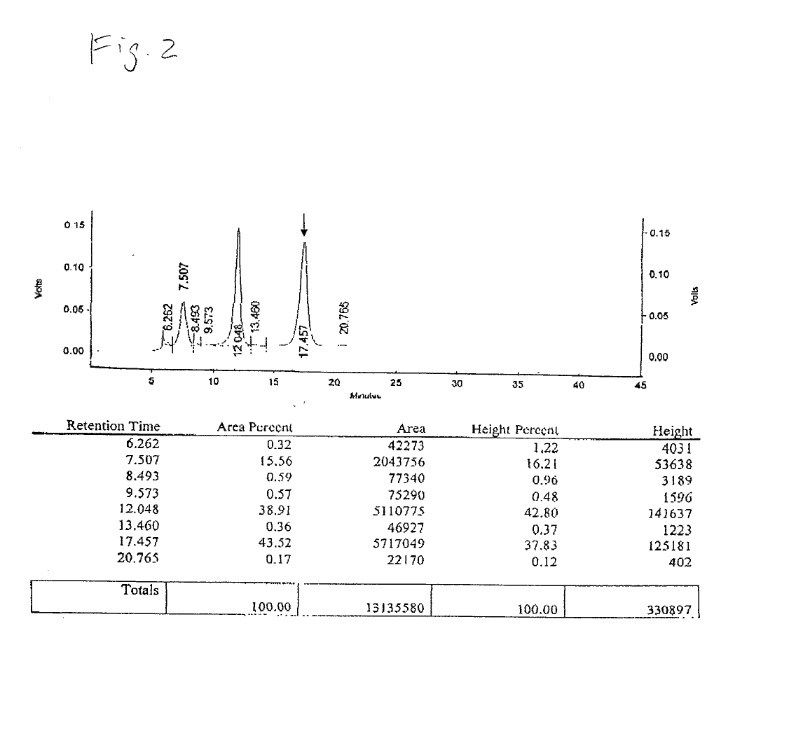

The result of HPLC determination of naringin content is shown is FIG. 2.

### EXAMPLE 3

### A Method for Preparation of Herba epimedium Extract.

Raw Material:

Herba Epimedii—It is the dried leaves of Epimedium brevicornum Maxim, Epimedium sagittatum (Sieb. Et Zucc) Maxim., Epimedium pubescens Maxim., Epimedium wushanense T. S. Ying or Epimedium koreanum Nakai, all of which are herbaceous perennial plants of Berberidaceae family. The plant grows mainly in Shianxi, Sichuan, Hubei, Shanxi and Guangxi provinces of China. The plant is harvested in summer or autumn, its leaves are collected, cleaned, dried and used for the extract preparation.

Extraction Process:

Cut raw herb into small pieces and prepare coarse herbal powder using grinder. Put the coarse powder into extractor, add 10 volume water (v/w), heat to boil and extracted twice, 1.5 hours for the 1^{st} time, and 1 hour the second. Combine the two extractions and filter.

Load the filtrate on a polyamide column of 30˜60 mesh. Polyamide was about 2 times of the herb (w/w). Wash the column with water till the elution becomes colorless. Elute the column with 50% alcohol. Start to collect the elution when alcohol is detected in the elution via an alcohol meter. Concentrate the elution by reducing pressure while have alcohol recovered. Evaporate the elution in water bath and have it dried in vacuum. Obtain condensed herbal extract.

Extract Specifications:

Herba epimedii extract is standardized and quality controlled to contain 10-20% of icariin (C₃₃H₄₀O₁₅) content by a methodology of HPLC.

Methodology of HPLC for icariin content test

Chromatographic system and system suitability test: use 18-alkyl silane bonded silica gel as the stationary phase and acetonitrile-water (30:70) as the mobile phase; The wavelength of the detector is 270 nm. The number of theoretical plates of the column is not less than 1500, calculated with the reference to the peak of icariin.

Preparation of reference solution: weigh accurately a certain amount of icariin standard, and add methanol to prepare a solution of 0.1 mg/ml.

Preparation of test solution: weigh 0.2 g herbal extract accurately and put it into a 50 ml volumetric flash, add 20 ml low proof alcohol accurately, then seal up and weigh. Weigh again after ultrasound treatment for 1 hour, and add the lost weight with additional low proof of alcohol. Shake well, filter and collect the filtrate.

Procedure: inject accurately 10 μl of each of the reference solution and the test solution into the HPLC column, determine and calculated the content.

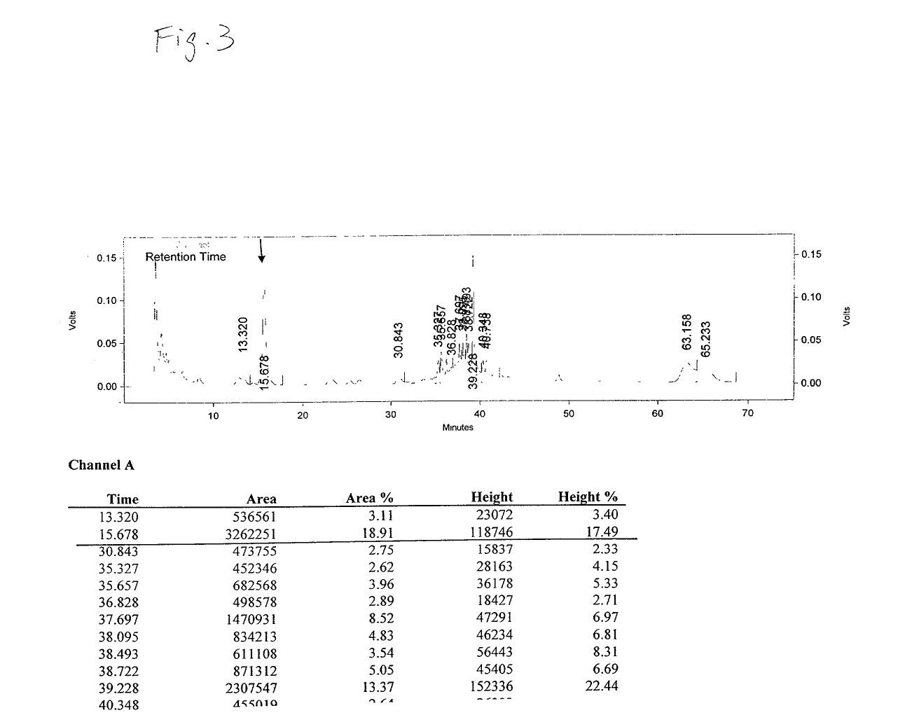

The result of HPLC determination of icariin content is shown in FIG. 3.

AHT-323 is a pharmaceutical preparation that contains Tripterygium hypoglaucum (L'evl.) Hutch extract, Rhizoma Drynariae extract and Herba epimedii extract. The preferred methods for preparing the three extracts are described above. It is also preferred that AHT-323 is prepared according the method below.

### EXAMPLE 4

### A Method for Preparation of the AHT-323

a. Blending

Combine the powder of the three single herbal extracts together with dextrin in the exact quantity and ratio as specified by the formula and specification described in Table 1 below, mix well and blend thoroughly.

TABLE 1

| | | | |
|---|---|---|---|
| Active Ingredients: | Tripterygii | 25 | mg |
| | hypoglaucum | | |
| | (L'evl.) Hutch | | |
| | Extracts | | |
| | Rhizoma | 25 | mg |
| | Drynariae | | |
| | Extracts | | |
| | Herba | 60 | mg |
| | epimedii | | |
| | Extracts | | |
| Formulation: | Active | 110 | mg |
| | ingredients | | |
| | Dextrin | 90 | mg |
| | Total | 200 | mg |

b. Encapsulation

Fill in each capsule with 200 mg of the mixture above.

c. Packaging

Fill HDPE bottles of 120 cc with 60 capsules each.

Specifications for the Preparations of the Herbal Mixture

Each capsule contains a total of 200 mg mixture of herbal extract and excipient. It is standardized and quality controlled to contain approximately 25 mg of Tripterygii hypoglaucum (L'evl.) Hutch extract, 25 mg of Rhizoma Drynariae extract, and 60 mg of Herba epimedii extract along with approximately 90 mg of dextrin. Each capsule contains approximately 8-10 μg triptolide.

Generally, AHT-323 may be orally administered to patients suffering from autoimmune diseases or inflammatory disorders at approximately 1-1.5 μg triptolide per kg body weight per day based on the following Table 2.

TABLE 2

| Body Weight | Dose of AHT-323 (Number of 200 mg Capsules) | | |
|---|---|---|---|
| (Kg) | AM | PM | Evening |
| 35 to <45 kg | 1 | 1 | 2 |
| 45 to <55 kg | 2 | 1 | 2 |
| 55 to <65 kg | 2 | 2 | 2 |
| 65 to <75 kg | 2 | 2 | 3 |

The drug can be used alone or along with steroid, immune suppressive agents and/or anti-inflammatory drugs depending the severity and activity of the disease.

Pharmacological studies were conducted in vivo and in vitro to assess the effects of AHT-323 on inflammation, immune response and pain.

AHT-323 showed significant inhibitory effects on inflammation. It suppressed the primary and secondary lesions in a rat model of adjuvant arthritis (AA). It inhibited inflammatory reaction and restrained capillary hyper-permeability of mice induced by acetic acid, inhibited mouse auricular swelling caused by croton oil, and rat paw swelling induced by Fucus siliquosus glue. It also prohibited migration of WBC of pleura in mice as well as WBC aggregation in CMC pouch of rats.

AHT-323 also exhibited significant suppressive effects on immune responses. It suppressed delayed type hypersensitivity of mouse ears induced by DNFB. It inhibited systemic immune responses of mice, including the formation of hemolysin antibody, the release of IL-1, IL-2, IL-6 and TNF, and the activities of macrophage and spleen cells. It inhibited lymphocyte blastogenesis of normal mice induced by Con A and the activity of NK cells while without causing atrophy of immune organs. AHT-323 was capable of normalizing abnormally increased CD4/CD8 ratio in AA rats at a dose-dependent manner.

In addition, AHT-323 revealed significant effects on pain relief in mice and blood rheology improvement in AA rats.

BALB/c mice were provided by Experimental Animal Center of West-China Medical University. NIH mice were provided by the Chengdu Institute of Biological Products. SD rats were provided by Sichuan Institute of Chinese Materia Medica.

### EXAMPLE 5

### Effect of AHT-323 on Adjuvant Arthritis of Rats^{[1]}

1. Prophylactic Effect on the Rat Adjuvant Arthritis

The study was conducted in adjuvant arthritis (AA) rats, an experimental model for human rheumatoid arthritis. The AA rats exhibit clinical symptoms and pathological changes in paw and ankle joints, which mimic joint inflammation and autoimmune reactions seen in human RA.

In the study, 60 male SD rats weighing 180-220 g were used and randomly divided into 5 groups, including three test groups, one negative and one positive control groups. The rats were given p.o. three different dosages of AHT-323, tragacanth, or prednisone, respectively, once daily for 4 consecutive weeks. 0.1 ml of Freund's Complete Adjuvant (FCA) was also injected intradermally into the plantar of left hind paw of rats once daily. Each day, the maximal circumference of both left and right paw and ankle joint were measured, and the swelling degrees (mm) were calculated to determine the AHT-323 on both primary and secondary injury. The primary injury occurs at the homolateral joints of the adjuvant injection and is resulted from direct, non-specific inflammatory reaction of the body to adjuvant. Whereas the secondary injury occurs at the contralateral joints of the adjuvant injection in about two weeks after adjuvant injection and is related to auto-immune responses induced by adjuvant injection. The pathological changes of human rheumatoid arthritis is similar to that of the secondary injury in AA rats.

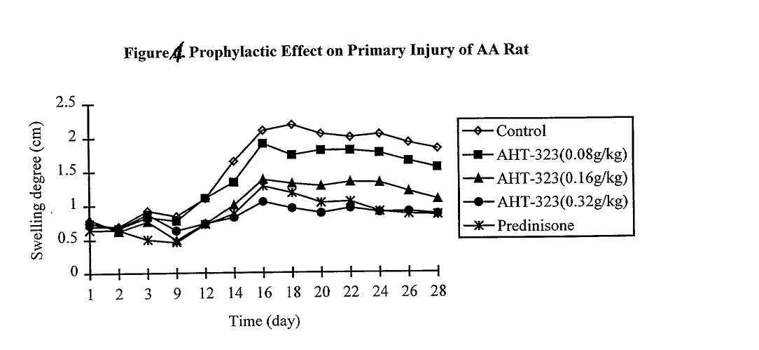

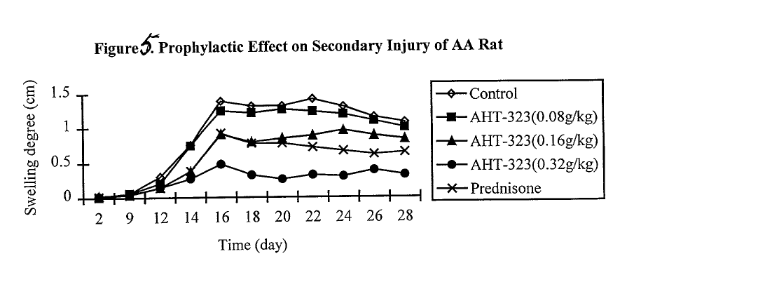

The results indicated that AHT-323 prohibited both the primary (left paw) and secondary (right paw) injuries of the AA rats in a dose-dependent manner when AHT-323 was introduced on day 1 of FCA injection. The inhibitory effects of AHT-323 at 0.32 g/kg exhibited about the same on primary injury while much stronger on the secondary injury than that of prednisone control at 0.01 g/kg in terms of onset time and intensity (Table 4 & FIG. 4, Table 5 & FIG. 5.)

TABLE 4
Prophylactic Effect of AHT-323 on Primary Injury of AA Rats ( X ± Sd)

| | dose | Swelling degree (cm) | | | | | | |
|---|---|---|---|---|---|---|---|---|
| Group | g/kg | d1 | d2 | d3 | d9 | d12 | d14 | d16 |
| Control | | 0.69 ± 0.17 | 0.69 ± 0.12 | 0.92 ± 0.18 | 0.84 ± 0.41 | 1.10 ± 0.30 | 1.65 ± 0.68 | 2.10 ± 0.55 |
| AHT-323 | 0.08 | 0.71 ± 0.12 | 0.69 ± 0.13 | 0.78 ± 0.26 | 0.75 ± 0.24 | 0.97 ± 0.36 | 1.10 ± 0.57 | 1.63 ± 0.61 |
| | 0.16 | 0.72 ± 0.11 | 0.68 ± 0.16 | 0.71 ± 0.18 | 0.66 ± 023 | 0.88 ± 0.27 | 1.03 ± 0.36* | 1.37 ± 0.33* |
| | 0.32 | 0.59 ± 0.17 | 0.57 ± 0.15 | 0.70 ± 0.26 | 0.52 ± 0.26* | 0.57 ± 0.27** | 0.56 ± 024** | 0.96 ± 0.45** |
| Prednisone | 0.01 | 0.64 ± 0.14 | 0.64 ± 0.16 | 0.50 ± 0.26* | 0.46 ± 0.25* | 0.72 ± 0.46* | 0.87 ± 0.46** | 1.28 ± 0.69* |

| | | | | | | | |
|---|---|---|---|---|---|---|---|
| Control | | 2.18 ± 0.44 | 2.05 ± 0.46 | 2.00 ± 0.46 | 2.04 ± 0.57 | 1.92 ± 0.65 | 1.83 ± 0.67 |
| AHT-323 | 0.08 | 1.59 ± 0.59 | 1.59 ± 0.61 | 1.54 ± 0.63 | 1.44 ± 0.64* | 1.36 ± 0.63* | 1.26 ± 0.56* |
| | 0.16 | 1.47 ± 0.43** | 1.50 ± 0.43** | 1.49 ± 0.43* | 1.42 ± 0.53* | 1.40 ± 0.56* | 1.32 ± 0.57 |
| | 0.32 | 0.91 ± 0.49** | 0.85 ± 0.49** | 0.87 ± .49** | 0.75 ± .55** | 0.75 ± 047** | 0.69 ± 0.51** |
| Prednisone | 0.01 | 1.18 ± 0.76** | 1.03 ± 0.67** | 1.05 ± 0.69** | 0.90 ± 0.64** | 0.86 ± 0.65** | 0.85 ± 0.59** |

Note: *P<0.05, **P<0.01 compared with control. N = 12

TABLE 5
Prophylactic Effect of AHT-323 on Secondary Injury of AA Rats ( X ± Sd)

| | Dose | Swelling degree (cm) | | | | | |
|---|---|---|---|---|---|---|---|
| Group | g/kg | d2 | D9 | d12 | d14 | d16 | d18 |

| | | | | | | | |
|---|---|---|---|---|---|---|---|
| Control | | 0.017 ± 0.039 | 0.058 ± 0.073 | 0.30 ± 036 | 0.76 ± 0.52 | 1.39 ± 0.67 | 1.32 ± 0.60 |
| AHT-323 | 0.08 | 0.29 ± 0.40 | 0.054 ± 0.58 | 0.18 ± 011 | 0.21 ± 0.26** | 0.95 ± 0.57 | 0.96 ± 0.63 |
| | 0.16 | 0.042 ± 0.047 | 0.054 ± 0.050 | 0.15 ± 0.066 | 0.18 ± 0.17* | 0.90 ± 0.53 | 0.88 ± 0.64 |
| | 0.32 | 0.012 ± 0.031 | 0.050 ± 0.043 | 0.14 ± 0.056 | 0.10 ± 0.14** | 0.47 ± 0.33** | 0.41 ± 0.38** |
| Prednisone | 0.01 | 0.016 ± 0.039 | 0.038 ± 0.048 | 0.14 ± 0.14 | 0.38 ± 0.39 | 0.92 ± 0.62 | 0.78 ± 0.71 |

| | | | | | | |
|---|---|---|---|---|---|---|
| Control | | 1.32 ± 0.60 | 1.42 ± 0.68 | 1.31 ± 0.74 | 1.16 ± 0.77 | 1.08 ± 0.62 |
| AHT-323 | 0.08 | 0.99 ± 0.62 | 1.07 ± 0.64 | 1.02 ± 0.67 | 0.84 ± 0.59 | 0.86 ± 0.51 |
| | 0.16 | 0.85 ± 0.61 | 0.93 ± 0.66 | 0.84 ± 0.62 | 0.86 ± 0.63 | 0.78 ± 0.65 |
| | 0.32 | 0.38 ± 0.40** | 0.37 ± 0.30** | 0.29 ± 0.31** | 0.24 ± 0.26** | 0.24 ± 0.24** |
| Prednisone | 0.01 | 0.78 ± 0.70 | 0.72 ± 0.71* | 0.67 ± 0.64* | 0.62 ± 0.61* | 0.65 ± 0.64* |

Note: *P<0.05, **P<0.01 compared with control. N = 12

2. Therapeutic Effect on the Adjuvant Arthritis Rats

Totals of 50 male SD rats were divided into 5 groups with the same treatment as experiments described in section 3.3.1 except that the AHT-323 was given p.o. from d13˜d28 once daily for 2 weeks, (from the thirteenth day after injection of Freund's Complete Adjuvant).

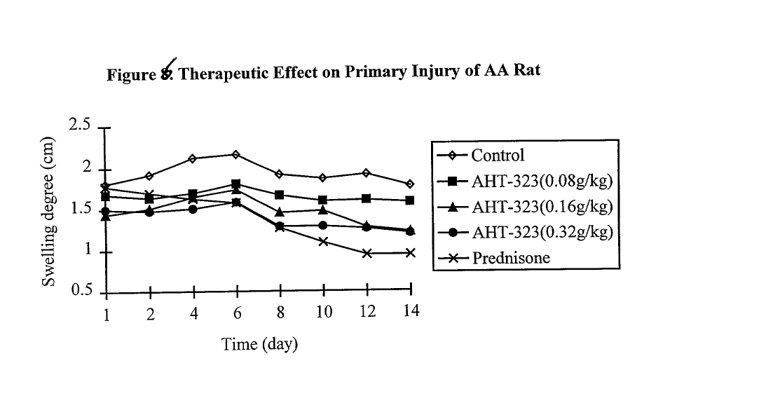

The results indicated that the test drug showed significant inhibition in the primary and secondary lesions of AA rats in a dose-dependent manner when AHT-323 was introduced on day 13 of FCA injection. The inhibitory effects of AHT-323 at 0.16 and 0.32 g/kg exhibited about the same on both the primary and secondary injuries as that seen in prednisone control at 0.01 g/kg in terms of onset time and intensity (Table 6, 7 and FIG. 6, 7).

TABLE 6
Therapeutic Effect of AHT-323 on Primary Injury of AA rats ( X ± Sd )

| | Dose | Swelling degree (cm) | | | |
|---|---|---|---|---|---|
| | (g/kg) | D1 | d2 | d4 | d6 |
| Control | | 1.81 ± 0.27 | 1.92 ± 0.19 | 2.12 ± 0.22 | 2.16 ± 0.22 |
| AHT-323 | 0.08 | 1.69 ± 0.42 | 1.62 ± 0.34 | 1.68 ± 0.48 | 1.74 ± 0.56 |
| | 0.16 | 1.58 ± 0.30* | 1.57 ± 0.33** | 1.60 ± 0.28** | 1.60 ± 0.33** |
| | 0.32 | 1.48 ± 0.60 | 1.46 ± 0.38** | 1.56 ± 0.42** | 1.58 ± 0.50** |
| Pre- | 0.01 | 1.78 ± 0.51 | 1.70 ± 0.51 | 1.63 ± 0.50* | 1.58 ± 0.50** |
| dnisone | | | | | |
| Control | | 1.92 ± 0.32 | 1.87 ± 0.34 | 1.92 ± 0.39 | 1.78 ± 0.44 |
| AHT-323 | 0.08 | 1.67 ± 0.68 | 1.60 ± 0.71 | 1.61 ± 0.77 | 1.58 ± 0.71 |
| | 0.16 | 1.49 ± 0.28** | 1.48 ± 0.30* | 1.42 ± 0.37** | 1.45 ± 0.49* |
| | 0.32 | 1.36 ± O.42** | 1.32 ± O.56** | 1.28 ± 0.56** | 1.22 ± 0.56** |
| Pre- | 0.01 | 1.27 ± 0.46** | 1.09 ± 0.54** | 0.94 ± 0.50** | 0.94 ± 0.42** |
| dnisone | | | | | |

Notes: *P<0.05, **P<0.01, compared with control. N = 10

TABLE 7
Therapeutic Effect on Secondary Injury of AA Rats ( X ± Sd)

| | Dose | Swelling degree (cm) | | | |
|---|---|---|---|---|---|
| Group | (g/kg) | D1 | d2 | d4 | d6 |
| Control | | 1.12 ± 0.45 | 1.48 ± 00.42 | 1.57 ± 00.38 | 1.67 ± 00.35 |
| AHT-323 | 0.08 | 1.06 ± 00.46 | 1.16 ± 00.48 | 1.36 ± 00.48 | 1.50 ± 00.62 |
| | 0.16 | 1.01 ± 00.47 | 1.04 ± 00.51* | 1.19 ± 00.49 | 1.38 ± 00.51 |
| | 0.32 | 0.98 ± 00.52 | 0.96 ± 00.62 | 1.16 ± 00.50 | 1.17 ± 00.44* |
| Pre- | 0.01 | 1.08 ± 00.41 | 1.18 ± 00.44 | 1.23 ± 00.42 | 1.19 ± 00.38** |
| dnisone | | | | | |
| Control | | 1.58 ± 00.29 | 1.60 ± 00.35 | 1.58 ± 00.23 | 1.52 ± 00.24 |
| AHT-323 | 0.08 | 1.42 ± 00.54 | 1.40 ± 00.62 | 1.36 ± 00.68 | 1.32 ± 00.69 |
| | 0.16 | 1.06 ± 00.50** | 1.14 ± 00.49* | 0.99 ± 00.48** | 0.86 ± 00.43** |
| | 0.32 | 1.00 ± 00.56** | 1.04 ± 00.68* | 0.92 ± 00.60** | 0.84 ± 00.64** |
| Prednisone | 0.01 | 0.88 ± 00.47** | 0.95 ± 00.43** | 0.82 ± 00.48** | 0.75 ± 00.37** |

Notes: *P<0.05, **P<0.0 compared with control. N = 10

3. Effect on Pathological Changes in AA Rats

Totals of 38 rats weighing 180±20 g were divided into 5 groups. The FCA was injected to establish the AA model and AHT-323 was administered. AHT-323 was given from d0 to d24 and the articular index was calculated at d24 based on sum of swelling degree of each leg of four limbs at a scale of 0-4 as the following standard: 0=normal, 1=mild red, 2=red and mild swelling, 3=severe swelling, and 4=deformity and stiff joint. The tissue slides of hind leg joint with secondary lesions were prepared, stained with HE staining, and evaluated for articular cartilage pathological changes.

The test drug significantly decreased articular index of AA rats (Table 8) and prevented the rats from pathological changes of secondary lesion (Data are not shown).

TABLE 8
Influence of AHT-323 on Articular Index of AA Rat (X ± Sd)

| Group | Dose (g/kg) | Number of rats | Articular index |
|---|---|---|---|
| Control | — | 8 | 0** |
| AA model | — | 7 | 6.29 ± 0.49 |
| AHT-323 | 0.08 | 9 | 4.86 ± 0.90** |
| AHT-323 | 0.16 | 7 | 4.71 ± 0.95** |
| AHT-323 | 0.32 | 7 | 4.56 ± 1.13** |

Notes:
**P < 0.01, compared with AA model group.

### EXAMPLE 6

### Effects of AHT-323 on Delayed Hypersensitivity of Mice Induced by DNFB^{[2]}

Totals of 50 NIH mice with equal sex were randomly divided into 5 groups. Each mouse was sensitized with 0.025 ml DNBF (1%) at day 0. Five days later (day 6), 0.01 ml DNFB was pasted as aggression on the right ear. The mice were sacrificed 24 hours later and the weight of left and right ears were measured, the difference of which was used to evaluate reaction intensity of the delayed type hypersensitivity (DTH) of the mice.

1. Effect on DTH When AHT-323 Was Given during Entire Course of DTH

In this experiment, AHT-323 was given for consecutive six days (0˜5 days) from the day when the mice were first sensitized with DNFB.

The result indicated that AHT-323 significantly inhibited DTH reaction of mouse ear caused by DNFB in a dose-dependent manner. (Table 9)

TABLE 9
Effect of AHT-323 on Mice Ear DTH Induced
by DNFB ( X ± Sd, N = 10)

| | Dose | Dosing | % of ear | Inhibition | P |
|---|---|---|---|---|---|
| Group | (g/kg) | (day) | Swelling | rate (%) | Value |
| Control | | | 15.4 ± 8.3 | | |
| AHT-323 | 0.08 | 0˜5 | 16.4 ± 6.8 | −6.5 | >0.05 |
| | 0.16 | 0˜5 | 12.4 ± 8.6 | 9.1 | >0.05 |
| | 0.32 | 0˜5 | 9.3 ± 4.5 | 39.6 | <0.05 |
| Cy | 0.020 | 0˜5 | 5.4 ± 2.6 | 64.9 | <0.01 |

Note: Cy = Cytoxan

2. Effect on DTH When AHT-323 Was Given at Different Intervals

Totals of 60 NIH mice were used. AHT-323 treatments were given at different intervals as the following: from d-2 (2 days before first sensitizing) to Day 0 (first sensitization day), from Day-2 to Day 2 (2 days after first sensitizing), from Day-2 to Day5, and from Day 0 to Day 4, respectively. The swelling rate of mouse ear was determined. The result indicated that AHT-323 inhibited significantly the DTH reaction indicated as swelling rate of mice ear at each of the treatment schedule. The treatment schedule from Day-2 to Day 5 showed most significant inhibition, suggesting that the mechanism of action by which AHT-323 inhibits DTH is by inhibition of cells that participate in the DTH reaction (Table 10).

TABLE 10
Effect of AHT-323 Given at Different Intervals
on DTH of Mice ( X ± Sd, N = 10)

| | Dose | Dosing | % of ear | Inhibition | P |
|---|---|---|---|---|---|
| Group | (g/kg) | (day) | Swelling | rate (%) | Value |
| Control | | | 46.2 ± 6.1 | | |
| AHT-323 | 0.32 | −2˜0 | 34.3 ± 6.9 | 25.8 | <0.01 |
| | 0.32 | −2˜2 | 30.4 ± 7.8 | 34.2 | <0.01 |
| | 0.32 | −2˜5 | 28.4 ± 6.8 | 38.5 | <0.01 |
| | 0.32 | 0˜4 | 32.9 ± 7.6 | 28.8 | <0.01 |
| Cytoxan | 0.025 | 0˜5 | 32.8 ± 7.9 | 29.0 | <0.01 |

Another 50 NIH mice were used to compare AHT-323 with cyclophosphamide or cytoxan (Cy) on the effects on DTH of mice. The mice were treated with either AHT-323 or Cy alone or both at different intervals. The results indicated that high dose of Cy given 3 days before sensitizing did not inhibit DTH reaction but rather enhance it. This may be due to the inhibitory effect of Cy on T suppressor cells (Ts) resulting in relative hyper-activity of T helper cells (Th). When AHT-323 was combined with Cy at the high dose, no synergetic effects were found (Table 11).

TABLE 11
Effect of AHT-323 and Cy on DTH of Mice (X ± Sd, N = 10)

| | Dose | Dosing | % of ear | Inhibition | P |
|---|---|---|---|---|---|
| Group | (g/kg) | (day) | Swelling | rate (%) | value |
| Control | | | 40.06 ± 5.02 | | |
| Cy | 0.1×3 | 0, 2, 4 each | 24.10 ± 7.28 | 39.8 | <0.01 |
| Cy | 0.25 | −3 | 44.3 ± 7.9 | −10.6 | >0.05 |
| AHT-323 | 0.32 | 0˜4 | 26.32 ± 7.65 | 34.3 | <0.01 |
| AHT + Cy | 0.32 + 0.25 | −3, 0˜4 | 39.44 ± 7.08 | 1.6 | >0.05 |

Note: Cy = cyclophosphamide

### EXAMPLE 7

### Effects of AHT-323 on Systemic Immune Responses^{[3]}

1. Effect on Hemolysin Antibody Formation in Normal Mice

Totals of 150 mice (50 NIH mice, 50 ICR mice and 50 KM mice) weighing 18-22 g with equal sex distribution were divided into 10 groups, each of which was immunized with 0.2 ml 5% sheep red blood cells (SRBC) i.p. once. AHT-323 was given from d0 to d6. Seven days after immunization, mice were sacrificed and blood was taken from the eyeball. Assay for hemolysin antibody formation was performed and its inhibition rate was evaluated. The results indicated that AHT-323 inhibited significantly the formation of hemolysin antibody in a dose-dependent manner (Table 12, 13 & 14).

TABLE 12
Effect on Hemolysin Antibody Formation
in NIH Mice (X ± S,d N = 10)

| | Dose | Dosing | Hemolysin | Inhibition | P |
|---|---|---|---|---|---|
| Group | (g/kg) | (day) | Antibody* | Rate (%) | Value |
| Control | | | 134 ± 84 | | |
| AHT-323 | 0.16 | 0˜7 | 51 ± 43 | 61.9 | <0.01 |
| AHT-323 | 0.32 | 0˜7 | 32 ± 21 | 76.1 | <0.01 |
| AHT-323 | 0.64 | 0˜7 | 25 ± 4 | 81.3 | <0.01 |
| Cy | 0.25 | 0˜7 | 33 ± 8 | 75.4 | <0.01 |

* HC₅₀

TABLE 13
Effect on Hemolysin Antibody Formation
in ICR Mice (X ± Sd, N = 10)

| | Dose | Dosing | Hymolysin | Inhibition | P |
|---|---|---|---|---|---|
| Group | (g/kg) | (day) | Antibody | Rate (%) | Value |
| Control | — | — | 124 ± 43 | | |
| AHT-323 | 0.08 | 0˜7 | 74 ± 52 | 40.3 | <0.05 |
| AHT-323 | 0.16 | 0˜7 | 53 ± 7 | 57.3 | <0.01 |
| AHT-323 | 0.32 | 0˜7 | 25 ± 5 | 79.8 | <0.01 |
| Cy | 0.02 | 0˜7 | 28 ± 7 | 77.4 | <0.01 |

Note: Cy = cytoxane. Control = 0.5% tragacanth

TABLE 14
Effect of AHT-323 Given in Different Intervals
on Hymolysin Antibody Formation in KM Mice (X ± Sd, N = 10)

| | Dose | Dosing | Hymolysin | Inhibition | P |
|---|---|---|---|---|---|
| Group | (g/kg) | (day) | Antibody | Rate (%) | value |
| Control | | | 243 ± 36 | | |
| AHT-323 | 0.16 | −7˜7 | 172 ± 44 | 29.2 | <0.01 |
| AHT-323 | 0.16 | −3˜7 | 162 ± 39 | 33.3 | <0.01 |
| AHT-323 | 0.16 | 0˜7 | 122 ± 35 | 49.8 | <0.01 |
| Cy | 0.02 | 0˜7 | 46 ± 18 | 81.1 | <0.01 |

Note: Cy = cyclophosphamide, Control = 0.5% tragacanth

The above tables showed that AHT-323 had significant inhibition effect on hemolysin antibody formation of different strains of mice (NIH mice and ICR mice and KM mice). The effect showed dose dependent correlation and the minimal inhibiting dose of AHT-323 was 0.08 g/kg.

2. Effect on Systemic Immune Response of AA Mice

An experimental model of adjuvant arthritis was established in NIH mice weighing 20±2 g by intradermal injection of 0.05 ml Freund's Complete Adjuvant (FCA) to the planter of hind paw. Three weeks later, the mice were treated with different dose of AHT-323 for 5 days. At the same time, they were sensitized with 0.5 ml 10% SRBC i.p. The mice were sacrificed 5 days later. The spleen cells were prepared and the plaque forming cells (PFC) were measured, the serum was prepared and IgM was determined.

The results showed that the PFC and IgM level were significantly higher in AA mice than in normal. AHT-323 decreased significantly the PFC and IgM level in the AA mice model (Table 15).

TABLE 15
Effect of AHT-323 on Humoral Immunity of AA Mice (X ± Sd)

| Group | Dose (g/kg) | N. | PFC (OD₄₁₅) | IgM (Log₂) |
|---|---|---|---|---|
| Control | — | 8 | 0.819 ± 0.013# | 6.875 ± 0.641 |
| AA model | — | 10 | 0.940 ± 0.019** | 7.700 ± 0.599* |
| AHT-323 | 0.08 | 8 | 0.834 ± 0.012**# | 6.875 ± 0.641# |
| AHT-323 | 0.16 | 8 | 0.834 ± 0.012**# | 6.750 ± 0.886# |
| AHT-323 | 0.32 | 8 | 0.830 ± 0.014**# | 6.375 ± 0.518## |
| Cy | 0.020 | 10 | 0.835 ± 0.015**# | 6.950 ± 0.597# |

Note:
*P < 0.05,
**P < 0.01, vs. control group.
#P < 0.05,
##P < 0.01, vs. AA model group.

### EXAMPLE 8

### Effects of AHT-323 on Cytokines^{[3]}

1. Effect on TNF α and IL-2 in Mice

Total of 50 ICR mice weighing 18˜22 g with equal sex were divided into 5 groups. The mice were administrated orally with different doses of AHT-323 or control (0.5% tragacanth) once daily for 10 days. 24 hours after the last dosing, the peritoneal macrophage suspensions were prepared to measure TNFα content and IL-2 activity.

Assay for TNF α:

An ELISA assay was performed using anti-TNFα monoclonal antibody. The TNFα concentration was calculated based on the OD value at 450 nm.

Assay for IL-2:

The IL-2 activity of macrophage was measured with IL-2 dependent CTLL cells by ELISA.

The result showed that AHT-323 significantly inhibited TNFα at the doses 0.08 g/kg or above. AHT-323 at the doses 0.08 g/kg or above also inhibited IL-2 activity significantly (Table 16).

TABLE 16
Effect of AHT-323 on TNF and IL-2 (X ± Sd, N = 10)

| Group | Dose (g/kg) | TNFα (pg/ml) | IL-2 (IU/ml) |
|---|---|---|---|
| Control | — | 87.8 ± 14.6 | 26.3 ± 4.2 |
| AHT-323 | 0.08 | 64.8 ± 12.2** | 18.9 ± 3.1** |
| AHT-323 | 0.16 | 58.3 ± 10.3** | 16.0 ± 2.9** |
| AHT-323 | 0.32 | 57.5 ± 11.0** | 19.5 ± 3.2** |
| Cy | 0.02 | 42.2 ± 9.6** | 10.1 ± 3.0** |

Notes:
*P < 0.05,
**P < 0.01, vs. control.
Cy = cytoxan,
Control = tragacanth.

2 Effect on IL-1 and IL-6

Totals of 50 NIH mice weighing 18-22g with equal sex distribution were divided into 5 groups and administered p.o. with different doses of AHT-323 or cyclophosphamide once daily for 10 days. 24h after the last dosing, mice were sacrificed and peritoneal macrophage and spleen cells suspension were prepared to measure IL-1 and IL-6 activity, respectively.

Method of testing IL-1: IL-1 activity in peritoneal macrophages was measured with radio-ELISA and the result was shown in Table 18.

Method of testing IL-6: IL-6 activity in splenocytes was measured with ELISA and the result was shown in table 18.

The results indicated that AHT-323 significantly inhibited IL-1 production from peritoneal macrophages and IL-6 production from splenocytes in a dose-dependent manner (Table 17).

TABLE 17
Effect of AHT-323 on IL-1 and IL-6. (X ± Sd)

| Group | Dose (g/kg) | N | IL-1 (ng/ml) | IL-6 (IU/ml) |
|---|---|---|---|---|
| Control | — | 10 | 78.7 ± 7.1 | 94.6 ± 6.8 |
| AHT-323 | 0.08 | 10 | 65.4 ± 6.4** | 79.5 ± 7.6** |
| | 0.16 | 10 | 57.6 ± 4.7** | 65.7 ± 4.9** |
| | 0.32 | 10 | 53.8 ± 4.9** | 59.8 ± 6.4** |
| Cy | 0.02 | 9 | 44.5 ± 7.7** | 49.6 ± 6.7** |

Note:
Cy = cytoxan,
**P < 0.01 vs. control.

### EXAMPLE 9

### Effect of AHT-323 on Plasma NO of AA Rats

60 rats weighing 160-200 g with equal sex distribution were divided into 6 groups. Rats in the control group were intradermally injected with 0.5 ml NS to the planter of hind paw, while rats in the testing groups and experimental model control groups were intradermally injected with 0.5 ml Freund's complete adjuvant (FCA) once daily. Eighteen days later, rats in the test groups were treated with AHT-323 once daily for 5 days. Rats in the negative control and experimental model control groups were given distilled water. Rats in the positive control groups were given Tripterygium wilfordii polyglycosidium (TWP) drug. One hour after the last dose, 2 ml blood was taken from the abdominal aorta, plasma was isolated and preserved at −70 degree centigrade.

Method of testing NO:

The NO in the plasma was measured according to protocol of NO measuring kit.

The results (Table 18) indicated that NO level of AA model was significantly higher than that of control. AHT-323 significantly decreased NO level of AA rats. TWP showed similar effect but was significantly weaker than AHT-323. TWP has been used as a therapeutic drug for RA in China for more than 10 years and is not contained in the AHT-323.

TABLE 18
Effects of AHT-323 on Plasma NO of AA Rat. (X ± Sd)

| Group | Dose (g/kg) | N | NO (μmol/L) | y (y = Lgx) |
|---|---|---|---|---|
| Control | — | 8 | 13.55 ± 1.11* | 1.131 ± 0.032 |
| AA model | — | 9 | 17.56 ± 4.15 | 1.235 ± 0.097 |
| AHT-323 | 0.08 | 7 | 9.83 ± 2.08**## | 0.985 ± 0.087 |
| | 0.16 | 7 | 10.12 ± 1.56**## | 1.001 ± 0.067 |
| | 0.32 | 7 | 10.70 ± 1.51**## | 1.026 ± 0.062 |
| TWP | 0.006 | 7 | 15.25 ± 3.48 | 1.173 ± 0.099 |

Notes:
*P < 0.05,
**P < 0.01, vs. AA model group.
##P < 0.01 vs. control.

### EXAMPLE 10

### Effect of AHT-323 on Cellular Immunity

1. Effect on Lymphocyte Blastogenesis of Normal Mice

50 NIH mice with equal sex distribution were randomly divided into 5 groups. The mice were treated p.o. with different doses of AHT-323 or positive control drug once daily for 10 days. Twenty four hours after the last dosing, mice were sacrificed. The splenocytes were obtained and Con A (Concanavalin) was used to stimulate lymphocytes for transformation. ³H-TdR adulteration method was used and the result was shown in table 20.

The results indicated that AHT-323 inhibited significantly lymphocyte blastogenesis stimulated by Con A (Concanavalin) in certain dose-dependent correlation (Table 19).

TABLE 19
Effect of AHT-323 on Lymphocyte Blastogenesis
of Mice (X ± Sd)

| Group | Dose (g/kg) | N | Cpm | Stimulate Index |
|---|---|---|---|---|
| Control | — | 10 | 20433 ± 3579 | 25.87 ± 3.06 |
| AHT-323 | 0.08 | 10 | 18038 ± 3359** | 17.11 ± 2.60** |
| | 0.16 | 10 | 12081 ± 1039** | 17.58 ± 4.37** |
| | 0.32 | 10 | 12535 ± 1370** | 16.07 ± 2.04** |
| Cy | 0.02 | 9 | 9922 ± 1145** | 13.06 ± 2.28** |

Notes:
**P < 0.01, vs. control

2. Effect on CD4, CD8 and NK Cells of Mice

Totals of 50 ICR mice weighing 18˜22 g with equal sex distribution were randomly divided into five groups. The mice were administrated orally with different doses of AHT-323 or positive control drug respectively, once daily for consecutive ten days. 24 hours after the last dosing, the mice were sacrificed and splenocyte suspension was prepared for measurement of CD4 and CD8 content and NK cells activity.

CD4 and CD8 were measured with Biotin-labeled ELISA and the result was shown in table 21. NK cells activity was measured with colorimetry and the result was shown in table 20.

The results (Table 20) indicated that AHT-323 inhibited both CD4 and CD8 activities in a dose-dependent manner while it did not show significant effect on the CD4/CD8 ratio. Cytoxan significantly inhibited both CD4 and CD8, especially CD8, which resulted in a significantly increased CD4/CD8 ratio. AHT-323 also inhibited significantly NK cells with no significant dose-dependent correlation; Cytoxan also inhibited NK cells and the activity was much stronger than that of AHT-323.

TABLE 20
Effect of AHT-323 on CD4, CD8 and NK cells (X ± Sd, N = 10)

| | Dose | | | | |
|---|---|---|---|---|---|
| Group | (g/kg) | CD4 (%) | CD8 (%) | CD4/CD8 | NK Activity |
| Control | — | 20.80 ± 2.94 | 14.80 ± 2.49 | 1.42 ± 0.18 | 40.13 ± 4.89 |
| AHT-323 | 0.08 | 19.00 ± 2.21 | 13.30 ± 2.00 | 1.42 ± 0.07 | 36.84 ± 5.26**## |
| | 0.16 | 16.25 ± 2.25** | 11.00 ± 2.45** | 1.47 ± 0.14 | 34.36 ± 3.38*## |
| | 0.32 | 14.11 ± 2.41** | 9.63 ± 2.42** | 1.46 ± 0.22 | 32.20 ± 2.00**## |
| Cy | 0.02 | 11.50 ± 2.50** | 4.10 ± 1.20** | 2.91 ± 0.53** | 23.10 ± 3.66** |

Note:
*P < 0.05,
**P < 0.01 vs. control.
Cy = cytoxan

3. Effect on T Cells of AA Mice

A total of 60 NIH mice weighing 20±2 g were randomly divided into 6 groups, among which four groups were injected intradermially with 0.05 ml FCA to induce AA model mice. The negative control group was injected intradermially with 0.05ml saline. The rats in AHT-323 test groups were administrated orally with AHT-323 once daily for consecutive five days. Five days later, peripheral blood was drawn from mice in all groups and stained with esterase. The percent of T cells (positive esterase stained cells) was calculated.

The above mice were anesthetized and spleens were removed and made into splenocytes suspension. CD4⁺ T cells and CD8⁺ T cells were assayed with flow cytometry and CD4⁺/CD8⁺ was calculated. The result was shown in table 22.

The results (Table 21) revealed that there was no significant difference among ANAE⁺ groups. In the AA model control group, the CD4 increased, CD8 decreased and CD4/CD8 ratio elevated significantly compared with normal. Both AHT-323 treatment and wilfordine kept those parameters at normal levels, which differed significantly from the AA model group. No significant difference was found between AHT-323 group and normal group (p>0.05).

TABLE 21
Effect of AHT-323 on T cells of AA Mice (X ± Sd, N = 8)

| | Dose | ANAE⁺ | CD₄⁺ | CD₈⁺ | |
|---|---|---|---|---|---|
| Group | (g/kg) | (%) | (%) | (%) | CD₄⁺/CD₈⁺ |
| Control | — | 50.60 ± 4.25 | 26.13 ± 1.16 | 15.56 ± 0.68 | 1.68 ± 0.03 |
| AA model | — | 49.00 ± 4.22 | 32.56 ± 2.87** | 13.59 ± 1.03** | 2.49 ± 0.16** |
| AHT-323 | 0.08 | 49.13 ± 4.03 | 27.30 ± 1.76## | 15.98 ± 1.11## | 1.71 ± 0.04## |
| | 0.16 | 49.31 ± 3.29 | 27.96 ± 1.67## | 16.23 ± 1.27## | 1.73 ± 0.05## |
| | 0.32 | 48.56 ± 3.23 | 26.75 ± 1.94## | 15.58 ± 1.29## | 1.72 ± 0.04## |
| Wilfordine | 0.012 | 48.88 ± 2.89 | 27.88 ± 1.99## | 16.33 ± 1.31## | 1.70 ± 0.03## |

Notes:
*P < 0.05,
**P < 0.01 compared with control.
#P < 0.05,
##P < 0.01 compared with AA model group.

### EXAMPLE 11

### Effect of AHT-323 on Phagocytosis of Peritoneal Macrophage of Mice

This experiment demonstrated that AHT-323 had no significant inhibition on phagocytosis of macrophages, which indicates that AHT-323 may have little effect on the non-specific immunity.

Totals of 50 NIH mice weighing 18-22 g with equal sex distribution were randomly divided into 5 groups. The mice were treated p.o. with different doses of AHT-323 or other positive control drug once daily for 7 days. 1 h after the last dose, mice were injected i.p. with 0.2 ml WBC of chick (10%). 4 h later, mice were sacrificed and the peritoneal fluid was obtained. Under microscope, the amount of macrophage that phagocytosed CRBC and the amount of CRBC phagocytosed by each macrophage were counted.

The results indicated that AHT-323 had no significant effect on phagocytic function of peritoneal macrophage (Table 22).

TABLE 22
Effect on CRBC Phagocytosis of Macrophages in Abdominal
Cavity of ICR Mice (X ± Sd, N = 10)

| Group | Dose (g/kg) | Phagocytic percent (%) | Phagocytic index |
|---|---|---|---|
| Control | — | 25.75 ± 9.40 | 1.28 ± 0.20 |
| AHT-323 | 0.16 | 33.20 ± 12.77 | 1.46 ± 0.36 |
| | 0.32 | 35.20 ± 10.16 | 1.21 ± 0.20 |
| | 0.64 | 27.78 ± 20.14 | 1.53 ± 0.32 |
| DXM | 0.005 | 8.33 ± 10.13* | 1.10 ± 0.18 |

Notes:
*P < 0.05, vs. control.
DXM = dexamethasone

### EXAMPLE 12

### Effect of AHT-323 on Hyper-Permeability of Peritoneal Capillary^{[4]}

The inflammation develops in three stages: capillary dilation with increased vascular permeability (early stage), leukocytes aggregating in the inflammation focus (middle stage), and tissues proliferating and inflammation barrier formation (late stage). This experiment demonstrated that AHT-323 had significant inhibition on the early stage of the inflammation development.

Totals of 90 NIH mice weighing 18-22 g with equal sex distribution were randomly divided into 9 groups. The mice in the test groups were treated p.o. with different doses of AHT-323 once or once daily for 3 days. DXM was given once daily for 3 days to the positive control group. 1 h after the last dose, mice were injected i.p. with HAC saline (0.7%) and i.v. with 0.1 ml/10g B. W. of Evans blue saline (0.5%). 30 min later, the mice were sacrificed and permeability of peritoneal capillary was measured with colorimetry.

The result (Table 23) showed that AHT-323 inhibited significantly the hyper-permeability of peritoneal capillary induced by acetic acid when the drug was given for consecutive 3 days.

TABLE 23
Effect on Hyper-permeability of Peritoneal Capillary Induced
By Acetic Acid in Mice (X ± S, N = 10)

| | | | Permeability of dye | |
|---|---|---|---|---|
| Group | Dose (g/kg) | Dosing | (OD₅₉₀) | P value |
| Control | — | — | 0.29 ± 0.13 | |
| AHT-323 | 0.32 | qd × 1 | 0.26 ± 0.14 | >0.05 |
| | 0.64 | qd × 1 | 0.25 ± 0.10 | >0.05 |
| | 1.28 | qd × 1 | 0.25 ± 0.09 | >0.05 |
| Control | — | — | 0.28 ± 0.15 | |
| AHT-323 | 0.32 | qd × 3 | 0.25 ± 0.12 | >0.05 |
| | 0.64 | qd × 3 | 0.18 ± 0.10 | <0.05 |
| | 1.28 | qd × 3 | 0.15 ± 0.13 | <0.05 |
| DXM | 0.15 | qd × 3 | 0.11 ± 0.07 | <0.01 |

### EXAMPLE 13

### Effect of AHT-323 on Migration of Inflammatory Cells of Pleura^{[5]}

A total of 50 NIH mice were randomly divided into five groups. 0.5% Evans blue saline was injected into caudal vein at 0.1 ml/10 g body weight. Then mice were lightly anesthetized with ether and injected with 0.03 ml of Fucus siliquosus glue into right thoracic cavity to induce inflammation. 4 h and 32 h after inflammation induction, the mice were sacrificed and 2 ml of thoracic cavity wash liquid was collected and used to count the WBC and measure the optical density (OD) at 600 nm. The result was shown in table 25, which indicated that AHT-323 had significantly inhibited the aggregation of WBC of pleura, especially to early stage of aggregation. The regression equation on 4 h was y=44.13-2.01x, r=0.9625.

This experiment demonstrated that AHT-323 had significant inhibiting effect on leukocyte aggregation during inflammation development (Table 24).

TABLE 24
Effect of AHT-323 on Migration of Inflammatory Cells of Pleura (X ± Sd, N = 8)

| | Dose | WBC count | | Permeable dye (OD) | |
|---|---|---|---|---|---|
| Group | (g/kg) | 4 h | 32 h | 4 h | 32 h |
| Control | — | 46.0 ± 6.9 | 16.0 ± 9.6 | 0.156 ± 0.066 | 0.109 ± 0.019 |
| AHT-323 | 0.16 | 26.8 ± 4.5* | 14.2 ± 8.0 | 0.121 ± 0.062 | 0.116 ± 0.031 |
| | 0.32 | 10.9 ± 4.0** | 17.3 ± 4.6 | 0.100 ± 0.048 | 0.153 ± 0.032 |
| | 0.64 | 8.0 ± 5.5** | 6.6 ± 4.7* | 0.129 ± 0.066 | 0.092 ± 0.051 |
| DXM | 0.05 | 12.7 ± 10.2** | 4.4 ± 4.0* | 0.085 ± 0.045 | 0.063 ± 0.017 |

Notes:
*P < 0.05,
**P < 0.01, vs. control.

### EXAMPLE 14

### Effect of AHT-323 on WBC Aggregation in CMC Pouch of Rats^{[6]}

A total of 64 SD rats weighing 150-180g with equal sex distribution were randomly divided into 8 groups. The rats were treated p.o. with different doses of AHT-323 once daily for 3 days. One day before treatment, 20 ml CMC (1%) was injected into an air pocket on the back of the rats. At 3.5 h and 7.5 h, 0.1 ml liquid was taken from the pocket and dyed using brilliant crystal blue PBS solution. The number of WBC in CMC pocket liquid was counted.

The result indicated that AHT-323 significantly inhibited aggregation of WBC in CMC of rats in a dose dependent manner (Table 25). It demonstrates that AHT-323 inhibited WBC aggregation during inflammation development.

TABLE 25
Effect of AHT-323 on WBC Counts in CMC of Rats (X ± Sd, N = 8)

| | |
|---|---|
| Dosing | WBC count (× 10⁷/L) |

| | | | |
|---|---|---|---|
| Group | (g/kg) | 3.5 hour | 7.5 hour |

| | | | |
|---|---|---|---|
| Control | — | 9.7 ± 4.2 | 57.7 ± 17.3 |
| AHT-323 | 0.16 × 1 | 8.5 ± 3.5 | 39.4 ± 16.5 |
| | 0.32 × 1 | 8.7 ± 7.3 | 35.3 ± 23.2 |
| | 0.64 × 1 | 6.6 ± 3.3 | 18.1 ± 8.6** |
| Control | — | 10.9 ± 6.7 | 35.6 ± 11.2 |
| AHT-323 | 0.16 × 3 | 15.4 ± 9.7 | 38.6 ± 15.5 |
| | 0.32 × 3 | 4.8 ± 3.4** | 18.4 ± 12.2** |
| | 0.64 × 3 | 3.0 ± 2.8** | 11.0 ± 9.2** |

Notes: **P < 0.01 vs. control

### EXAMPLE 15

### Effect of AHT-323 on Ear Swelling Induced by Croton Oil^{[7]}

A total of 60 NIH mice weighing 18-22 g with equal sex distribution were randomly divided into 6 groups. The mice were treated p.o. with different doses of AHT-323, tragacanth, or DXM once daily for 3 days. One hour after the last administration, 0.02 ml croton oil (2%) was pasted around the left auricle. Four hours later, mice were sacrificed by dislocating cervical vertebrae. The right and left ears were cut off and weighed to get the differences in the degree of ear swelling.

The result demonstrated that AHT-323 inhibited significantly ear swelling induced by croton oil in a dose-dependent manner (Table 26).

TABLE 26
Effect on Paw Swelling Induced by Croton Oil (X ± Sd, N = 10)

| | Dose | Swelling degree | Inhibition | |
|---|---|---|---|---|
| Group | (g/kg) | (mg) | rate | P value |
| Control | — | 44.38 ± 9.40 | — | |
| AHT-323 | 0.16 | 39.05 ± 12.33 | 12.00 | >0.05 |
| | 0.32 | 36.65 ± 5.83 | 17.64 | <0.05 |
| | 0.64 | 34.91 ± 9.71 | 21.34 | <0.05 |
| DXM | 0.003 | 14.13 ± 5.75 | 68.16 | <0.01 |

### EXAMPLE 16

### Effect of AHT-323 on Paw Swelling Induced by Carrageenine in Rat^{[8]}

A total of 50 SD rats weighing 100-120g with equal sex distribution were randomly divided into 5 groups. The rats were treated p.o. with different doses of AHT-323, tragacanth, or aspirin once daily for 3 days. One hour later, rats were given subcutaneously injections with 0.05 ml of carrageenin (1%) in the right paw of each rat. The degree of paw swelling was measured using the drainage method before and 1, 3, 4, 5, and 6 h after injection of the carrageenin.

The result showed that AHT-323 significantly inhibited paw swelling of rats induced by carrageenin (Table 27).

TABLE 27
Effect on Paw Swelling Induced by Carrageenin (X ± Sd, N = 10)

| | | Paw swelling rate (%) | | | | |
|---|---|---|---|---|---|---|
| Group | (g/kg) | 1 h | 3 h | 4 h | 5 h | 6 h |
| Control | — | 13.0 ± 4.5 | 22.1 ± 5.2 | 29.3 ± 5.1 | 33.8 ± 6.1 | 35.9 ± 6.4 |
| AHT-323 | 0.16 | 10.4 ± 4.3 | 24.8 ± 8.1 | 33.4 ± 7.9 | 34.5 ± 9.9 | 33.7 ± 10.2 |
| | 0.32 | 9.1 ± 3.3 | 16.8 ± 5.7 | 26.6 ± 5.5 | 28.0 ± 4.3 | 30.6 ± 6.2 |
| | 0.64 | 7.0 ± 6.0* | 12.3 ± 5.8** | 13.8 ± 8.2** | 17.8 ± 8.2** | 23.2 ± 11.9* |
| Aspirin | 0.25 | 6.3 ± 4.1** | 11.1 ± 6.3** | 17.9 ± 7.6** | 18.1 ± 7.0** | 20.6 ± 6.0** |

Notes:
*P < 0.05,
**P < 0.01 vs. control

### EXAMPLE 17

### Effect of AHT-323 on Cotton Granuloma^{[9]}

150 SD rats weighing 150-180 g with equal sex distribution were randomly divided into 15 groups. After light anesthesia with ether, an aseptic cotton ball (10 mg) was implanted into each armpit. On that day, rats were administered p.o. with different doses of AHT-323 or DXM once daily for 7 days. Twenty four hours after the last drug administration, the rats were sacrificed and the cotton balled granuloma were removed. The wet and dry weight of the granuloma, and other organs were obtained.

AHT-323 showed significant effect on granuloma dry weight only at high doses. It showed no significant effect on the weight of other organs (Table 28). It indicated that AHT-323 significantly decreased the dry weight of granuloma but did not cause atrophy of other organs.

TABLE 28
Effect on Weight of Granuloma and Organs (X ± Sd, N = 10)

| | | Granuloma Wt. | | | | | | |
|---|---|---|---|---|---|---|---|---|
| | Dose | (mg/100 g) | | Weight of organs (mg/100 g) | | | | |
| Group | (g/kg) | Wet Wt. | Dry Wt. | Thymus | Spleen | Adrenal | testes | uterus |
| Control | — | 484 ± 36 | 110 ± 10 | 176 ± 44 | 818 ± 165 | 21 ± 7 | 1.34 ± 0.17 | 147 ± 14 |
| AHT- | 0.32 | 500 ± 70 | 108 ± 13 | 172 ± 64 | 681 ± 206 | 24 ± 6 | 1.41 ± 0.08 | 110 ± 29 |
| 323 | 0.64 | 453 ± 87 | 99 ± 16 | 178 ± 40 | 680 ± 230 | 28 ± 8 | 1.55 ± 0.11 | 155 ± 29 |
| | 1.28 | 460 ± 63 | 99 ± 13* | 142 ± 39 | 712 ± 214 | 26 ± 9 | 1.27 ± 0.05 | 146 ± 29 |
| DXM | 0.05 | 346 ± 92** | 77 ± 17** | 38 ± 13** | 412 ± 173** | 17 ± 5 | 1.48 ± 0.07 | 126 ± 33 |

Notes:
W. = weight.
*P < 0.05,
**P < 0.01, vs. control.
DXM = dexamethasone.

### EXAMPLE 18

### Effect of AHT-323 on Writhing Reaction^{[10]}

A total of 50 Kunming mice weighing 18-22 g with equal sex distribution were randomly divided into 5 groups. The mice were treated p.o. with different doses of AHT-323, tragacanth, or Morphine hydrochloride once daily for 3 days. One hour later, each mouse was treated with 0.2 ml saline containing 0.7% HAC, i.p. The time to first writhing and the number of writhing during the first 20 minutes were evaluated.

The result (Table 29) showed that AHT-323 significantly decreased the number of writhing within 20 minutes at high doses, indicating that the drug has some analgesic effect.

TABLE 29
Effect of AHT-323 on Writhing Reaction of Mice (X ± Sd, N = 10)

| | Dose | | Latent time |
|---|---|---|---|
| Group | (g/kg) | Torsion number | (min) |
| Control | — | 34.6 ± 14.1 | 3.13 ± 0.80 |
| AHT-323 | 0.32 | 28.2 ± 5.76 | 3.82 ± 0.85 |
| | 0.64 | 31.0 ± 18.4 | 3.86 ± 2.00 |
| | 1.28 | 20.7 ± 12.3* | 3.95 ± 1.42 |
| Morphine | 0.01 | 0.0 ± 0.0 | — |

### EXAMPLE 19

### Effect of AHT-323 on Blood Rheology of AA Rats

RA patients show abnormal changes in rheology particularly with whole blood viscosity, plasma viscosity, erythrocytes aggregation increases and erythrocytes packed cell volume decrease. Among them, the change of plasma viscosity is the most important and is considered as a marker index for status of illness and treatment. This experiment showed that AHT-323 significantly decreased the whole blood viscosity, plasma viscosity and increased erythrocytes packed cell volume in AA rats.

SD rats weighing 180±20 g were intradermially injected with 0.05 ml FCA for three weeks. Rats were divided into AA model group, AHT-323 treatment groups (low, medium and high dosage), and positive group given with wilfordine, and negative control group. Rats in negative control group were intradermially injected with 0.05 ml saline. AHT-323 treatment groups and positive control group were administered p.o. with AHT-323 or wilfordine once daily for 5 days. lh after the last dosing, 3 ml of blood was collected from abdominal aorta, and whole blood viscosity was determined at 230S⁻¹, 115S⁻¹, 46S⁻¹, 23S⁻¹, 11.5S⁻¹, and 5.75S⁻¹. Packed cell volume was measured by hematocrit, and then the rigid index and agglutinate index of red blood cell were determined.

The results showed that AA rats manifested abnormal blood rheology, such as increased plasma viscosity, decreased packed cell volume, increased hemagglutination index and rigid index. AHT-323 improved significantly the above blood rheology indexes (Table 30).

TABLE 30
Effect of AHT-323 on Blood Rheology of AA Rat (X ± Sd, N = 10)

| | | | AHT-323 (g/kg) | | | TWP(g/kg) |
|---|---|---|---|---|---|---|
| Group | Control | AA Model | 0.16 | 0.32 | 0.64 | 0.06 |
| WBV(mPa · s) | | | | | | |
| 230S⁻¹ | 4.43 ± 0.09 | 4.92 ± 0.15** | 4.56 ± 0.09## | 4.49 ± 0.l1## | 4.54 ± 0.16## | 4.66 ± 0.28# |
| 115S⁻¹ | 5.17 ± 0.25 | 5.81 ± 0.19** | 5.33 ± 0.09## | 5.32 ± 0.l0## | 5.16 ± 0.14## | 5.60 ± 0.48# |
| 46S⁻¹ | 6.84 ± 0.11 | 7.20 ± 0.18** | 6.56 ± 0.13## | 6.59 ± 0.09## | 6.67 ± 0.14## | 6.70 ± 0.48# |
| 23S⁻¹ | 8.10 ± 0.15 | 8.23 ± 0.38 | 7.95 ± 0.22 | 7.93 ± 0.12 | 7.97 ± 0.14 | 8.02 ± 0.14 |
| 11.5S⁻¹ | 9.35 ± 0.08 | 9.78 ± 0.10** | 9.40 ± 0.08## | 9.45 ± 0.10## | 9.30 ± 0.13## | 9.31 ± 0.12## |
| 6.5S⁻¹ | 11.03 ± 0.14 | 12.66 ± 0.31** | 11.21 ± 0.21## | 11.29 ± 0.19## | 11.06 ± 0.40## | 11.42 ± 0.52## |
| PV (mPa · s) | 1.158 ± 0.032 | 1.248 ± 0.040** | 1.161 ± 0.011## | 1.154 ± 0.023## | 1.156 ± 0.018## | 1.158 ± 0.029## |
| EPCV (%) | 46.13 ± 2.31 | 41.33 ± 1.13** | 45.10 ± 2.39## | 44.33 ± 1.52## | 45.71 ± 1.04## | 46.03 ± 3.59## |
| EAI | 2.49 ± 0.032 | 2.58 ± 0.083* | 2.46 ± 0.066# | 2.49 ± 0.094# | 2.44 ± 0.048## | 2.45 ± 0.091# |
| ERI | 6.155 ± 0.536 | 7.127 ± 0.557** | 6.506 ± 0.558 | 6.525 ± 0.146 | 6.394 ± 0.200# | 6.621 ± 0.883 |

Notes:
*P < 0.05,
**P < 0.01, compared with control.
#P < 0.05,
##P <0.01, compared with AA model group.
WBV = whole blood viscosity,
PV = plasma viscosity,
EPCV = erythrocyte packed cell volume,
EAI = erythrocyte agglutination index,
ERI = erythrocyte rigid index.

The following references further explain the above described examples and are incorporated by reference in their entirety.

### REFERENCES

1 Ward J R and Jones R S. Studies on adjuvant-induced polyarthritis in rat. Arthritis Rheum, 1962;5:557-564

2 Corsini A C, Bellucci S B, Costa M G. A simple method of evaluating delayed type hepersensitivity in mice. J Immunol Methods, 1979;30:195

3 Luqmani R; Sheeran T; Robinson M; et al. Systemic cytokine measurements: their role in monitoring the response to therapy in patients with rheumatoid arthritis. Clin Exp Rheumatol, 1994; 12(5):503-8

4 Whittle B A. The use of changes in capillary permeability in mice to distinguish between narcotic and nonnarcotic analgesics. Brit J Pharmacol, 1964;22:246-253

5 Henriques M G M O, Weg V, Martins M A, et al. Differential inhibition by two hetrazepine PAF antagonists of acute inflammation in the mouse. Bri J Pharmacol, 1990;99:164-168

6 Yoshino S ; Cromartie W J ; Schwab J H. Inflammation induced by bacterial cell wall fragments in the rat air pouch. Comparison of rat strains and measurement of arachidonic acid metabolites. Am J Pathol, 1985;121(2):327-36

7 Jones L H ; Abdalla D S ; Freitas J C. Effects of indole-3-acetic acid on croton oil- and arachidonic acid-induced mouse ear edema. Inflamm Res, 1995;44(9):372-5

8 Vinegar R, Schreiber W and Hugo R. Biphasic development of carrageenin edema in rats. J Pharmacol Exp Therap, 1969;160:96-103

9 Swingle K F and Shideman F E. Phases of the inflammatory response to subcutaneous implamtation of a cotton pellet and their modification by certain anti-inflammatory agents. J Pharm Exp Therap, 1972;183:226-234

10 Collier H O J. The abdominal constriction response and its suppression by analgesic drugs in the mouse. Brit J Pharmacol, 1968;32:295-310

### EXAMPLE 20

### Clinical Study of AHT-323

1. Study Design

This study is designed as a 12-week, randomized, double-blind, placebo-controlled, parallel Phase II trial comparing AHT-323 at three dosage levels (low, medium, and high) with placebo in patients with active RA. It will determine the effective dose range of AHT-323 for treating the condition during the 12-week treatment period. Patients with RA will be screened for their qualification for the study and those who meet the protocol entry criteria will be discontinue therapy with antiinflammatory, analgesic agent and other treatment at the start of a two-week placebo run-in period. Patients who qualify for the study will be randomized to one of four treatment groups (placebo, three doses of AHT-323) at the conclusion of the placebo run-in period. Treatment will be given for six weeks, following which patients will be returned to standard treatment regimens as appropriate.

At Visit 0 (screening), patients will be required to provide informed consent and will begin the two-week placebo run-in period. During the two-week placebo run-in period, patients will be required to undergo clinical variable baseline assessment for rheumatoid arthritis at Visit 1 and 2 to evaluate whether they meet the inclusion criteria for the study.

At the end of Visit 2, patients who meet the study eligibility criteria will be randomized to one of four treatment groups with AHT-323 at 200 mg tid, 600 mg tid, and 1,200 mg tid or placebo. Assigned study drug or placebo will be taken with the morning, noon and evening meals. Patients will be scheduled for six visits (once per week) during the double-blind treatment period to assess safety and efficacy of the study drug.

For safety assessment, patients will be monitored for vital signs, adverse reactions and serious adverse events during the six-week treatment period. The efficacy will be assessed based on the significant changes in joint swelling/pain, morning stiffness, grip strength and time required for walking 50 feet after treatment. Laboratory tests will also be conducted to assess changes in conversion rate of rheumatoid factor (RF) from positive to negative, changes in erythrocyte sedimentation rate (ESR) and in C-reactive protein (CRP) as well as changes in immunological responses indicated by C₃, C₄, IgA, IgG and IgM.

2. PATIENT SELECTION

2.1 Number of Patients

280 patients will be entered into the placebo run-in period to obtain 224 completed evaluable patients (56 in each treatment group) following randomization, assuming a 20% drop out rate. A completed patient is a patient who has been randomized and received one or more doses of study drug.

2.2. Inclusion Criteria

2.1.1 Eligibility

A patient must meet each of the following inclusion criteria to be eligible for randomization to study treatment:

a) Age 21 years or older;

b) Males, or females who use an adequate form of birth control;

c) Diagnosis of RA in a flare state (defined below), with a ARA functional capacity classification of I-III (defined below);

d) Ability to understand and willingness to provide written informed consent at the start of the placebo run-in period.

2.2.2 RA Flare State

A RA flare state will be considered to have occurred if the first 2, and either the third or the fourth, of the following criteria are met at baseline assessment:

a) A minimum of 6 tender joints and an increase in the number of tender or painful joints of 20% or at least 2 joints during the last one or two weeks;

b) A minimum of 3 swollen joints and an increase in the number of swollen joints of 20% or at least 2 joints since screening;

c) A minimum of 45 minutes of morning stiffness and an increase in morning stiffness of at least 15 minutes since screening;

d) Patient-assessed arthritis pain of at least 40 mm on the VAS and an increase of at least 20% or 10 mm since screening.

e) After demonstration of a symptom flare (to be verified by the investigators at the individual study sites), patients will be randomized into the four groups.

2.2.3 Definitions of four classes of A.R.A. Functional Capacity:

Class I: Complete functional capacity with ability to carry on all usual activities (i.e., of usual occupation).

Class II: Functional capacity adequate to conduct usual activities despite handicap of discomfort or limited mobility of one or more joints.

Class III. Functional capacity adequate to perform only little or none of the activities of usual occupation or self-care.

Class IV: Largely or wholly incapacitated with patient bedridden or confined to wheelchair, permitting little or no self-care.

2.3 Exclusion Criteria

A patient will be excluded from randomization to study treatment for any one of the following reasons:

a) RA functional class IV;

b) Diagnosis of any inflammatory condition other than RA or any noninflammatory type of arthritis symptomatic enough to interfere with the assessment of treatment efficacy;

c) Having recently begun receiving or having a change in regimen of any disease-modifying antirheumatic drugs, antimalarial agents, or glucocorticoids; or having taken any NSAIDs within the 2 days prior to the baseline visit (except aspirin ≦325 mg/day) or any analgesics within the 24 hours prior to the baseline visit; or having anticipated requirement for treatment with glucocorticoids or nonsteroidal antiinflammatory drug (NSAID) or any analgesics.

d) History of clinically significant renal disease, hepatic disease, or metabolic diseases and evidence of clinically significant renal disease (serum creatinine level more than 50% above the upper limit of normal), hepatic disease (Aspartate transaminase (AST) activity and/or Alanine transaminase (ALT) activity more than 3 times the upper limit of normal), or metabolic diseases (e.g. diabetes requiring insulin therapy or fasting glucose levels more than 50% above the upper limit of normal) at screening;

e) Presence of any clinically significant heart diseases and uncontrolled hypertension (systolic blood pressure >200 mm Hg and/or diastolic blood pressure >95 mm Hg;

f) Known intolerance to AHT-323 or components of the study drug;

g) Inability to take oral medication;

h) Use of an investigational drug within 30 days prior to the Visit;

i) Patient is a pregnant, plan to pregnant in next 12 months or a nursing mother;

j) Patient is sufficiently unstable that surgery or death is likely to occur during the study period;

k) Patient is taking any other herbal dietary supplements that may interfere with the assessment of treatment efficacy or safety;

l) Patient requires regular or chronic aspirin administration;

m) Any other conditions that in the opinion of the investigator would make participation in the study potentially harmful to the patient.

3. MEDICATION ADMINISTRATION

All patients who meet the protocol eligibility criteria at the screen visit (Visit 0) will 924 receive placebo three times daily, once in the morning, midday (12 noon) and evening during the initial placebo two-week run-in period. The first dose of placebo will be administered in the investigators' office at Visit 0. This dose should be administered by the patient at the same times of the day during each day of his or her participation in the study.

Following the completion of the two-week run-in period, each patient who remains eligible for randomization will be randomized to receive 6 weeks of treatment with AHT-323 at a dosage of 200 mg, 600 mg, 1200 mg t.i.d. or placebo, t.i.d. which will be matched using double-dummy methods. The number and frequency of administration of study drug capsules (active and/or placebo) will be identical during the placebo run-in and treatment periods as well as between different treatment groups (Table 31).

TABLE 31
Study Group and Dosing Regimens

| | | No. of Capsules at Each Dosage | | |
|---|---|---|---|---|
| | | No. of | No. of | Total |
| Group | Daily Dosage | AHT-323 | Dummy | Capsules |
| 1. | 200 mg t.i.d. | 1 | 5 | 6 |
| 2. | 600 mg t.i.d. | 3 | 3 | 6 |
| 3. | 1,200 mg t.i.d. | 6 | 0 | 6 |
| 4. | Placebo | 0 | 6 | 6 |

The number and frequency of administration of study drug capsules (active and/or placebo) will be identical during the placebo run-in and randomized treatment periods as well as between different treatment groups. All patients participating in the study will receive placebo three times daily during the two-week placebo run-in period. After completion of the placebo run-in period, patients who meet all of the protocol eligibility criteria will be randomized and study dosing will be performed by administration as follows:

Group 1: Patients will receive a total of 600 mg AHT-323 daily or 200 mg, t.i.d.

Group 2: Patients will receive a total of 1,800 mg AHT-323 daily or 600 mg, t.i.d.

Group 3: Patients will receive a total of 3,600 mg AHT-323 daily or 1,200 mg, t.i.d.

Group 4: Patients will receive placebo, t.i.d.

Patients will be instructed to discontinue use of other anti-inflammatory and/or analgesics prior to starting the study (at Visit 0). All concomitant medications will be recorded on the Case Report Form (CRF) along with dosing information, date(s) of administration, and indication(s) for use.

4. CLINICAL EVALUATIONS

4.1 Medical History

Medical history including years of RA will be obtained at Visit 0 (screening) and will be recorded on the appropriate page of the CRF.

4.2 Physical Examination

A physical examination will be performed at Visit 0 (screening), Visit 6 and at Visit 8 (endpoint) or Visit 9 (early termination); any clinically relevant changes from screening in physical examination will be recorded on the CRF.

4.3 Body Weight

Body weight will be recorded at each visit while height will be recorded at Visit 0 only.

4.4 Vital Signs

Vital signs, including sitting systolic blood pressure, sitting diastolic blood pressure, and sitting heart rate, will be recorded at every visit.

4.4.1 General and Specific Laboratory Testing

Patients will have blood drawn for laboratory testing at Visits 0, 2, 4, 6, and 8 (or 9 in case of early termination). Laboratory tests include the following:

Chemistry tests (SMA 20) including electrolytes, calcium, phosphate, magnesium, creatinine, blood urea nitrogen (BUN), total protein, albumen, alkaline phosphatase, gamma-glutamyl transferase (GGT), bilirubin (total and direct), aspartate transaminase (AST), alanine transaminase (ALT), lactic dehydrogenase (LDH), cholesterol, triglycerides, and glucose;

Complete blood count (CBC) with differential

Urinalysis

Specific laboratory tests are described in the section 6.4.10- 6.4.13.

4.4.2 Joint Assessment

Joint assessment will be performed at each visit to determine the joint count for tenderness and swelling.

1) Joint count for tenderness on pressure and/or pain on motion will be recorded and graded on a scale of 0=none, 1=positive response on questioning, 2=spontaneous response elicited, 3=withdrawal by patient on examination for 60 diarthrodial joints;

2) Joint count of swollen will be recorded and graded on a scale of 0=none, 1=detectable synovial thickening without loss of bony contours, 2=loss of distinctness of bony contours, and 3=bulging synovial proliferation with cystic characteristics for 58 joints.

4.4.3 Grip Strength

Grip strength will be assessed at each visit using an ordinary mercury-column sphygmomanometer. With the mercury column vertical and in full view, and with patient arm unsupported, the patient will be required to squeeze the cuff as hard as possible. The mercury height at the level maintained by squeezing, i.e., not at the initial bounce will be recorded. Give each hand three tries and record all three readings in sequence, even if one of them seems aberrant. Use the same hand throughout the study.

4.4.4 Time Required to Walk 50 Feet

The assessment will be performed at each visit and will be measured in seconds. The patient will be required to walk a straight, continuous distance as fast as possible (without running) from a standing start. The time at 25 feet and at 50 feet will be recorded. State if a walking aid is used; if so, specify.

4.4.5 Patient and Investigator Global Assessments

Global assessments of therapy will be conducted by patients and investigator at Visit 2 and each of the visits thereafter and will be recorded as change from baseline.

The patients' global assessments will be based on the answers the patients give to the question: “How do you rate your well being, in particular with regard to your morning stiffness and severity of joint pain relative to the time before this study: worse than before the study, as good as or bad as before the study, a little better than before the study, quite a bit better than before the study, much better than before the study.

The physician's global assessment will be based on the answer to the request: Please rate your patient's state of health, in particular regarding his/her rheumatoid arthritis on the following five point rating scale: worse than at baseline, same as at baseline, mild improvement from baseline, moderate improvement from baseline, and marked improvement from baseline, with baseline defined as the time of screening.

4.4.6 Measurement of the Erythrocyte Sedimentation Rate

Westergren method is used. Result of one-hour reading will be recorded. The test will be performed at Visit 2, 4, 6, and 8 (or 9 in case of early termination).

4.4.7 Measurement of the C-Reactive protein Level in Blood Samples

Patients have blood drawn for the c-reactive protein testing at visit 2, 4, 6, and 8 (or 9 in case of early termination).

4.4.8 Measurement of Igg, Iga, Igm, and complement c3 and c4 levels

Patients have blood drawn for the Igg, Iga, Igm, and complement c3 and c4 testing at visit 2, 4, 6, and 8 (or 9 in case of early termination).

4.4.9 Measurement of sIL-2R, sIL-6R, and sTNF-R1

Patients have blood drawn for sIL-2R, sIL-6R and sTNF-R1 testing at visit 2, 4, 6, and 8 (or 9 in case of early termination).

5. Study Visits

Schedule of visits during the clinical trial and the activities conducted in each visit are summarized in Table 32, and explained as follows.

5.1 Screening

At visit 0 (screening), after signing an informed consent form, patients will undergo the following procedures:

complete medical history

physical examination

height and body weight

vital signs

baseline joint assessment

grip strength

time required for 50 feet

general laboratory tests (SMA20, CBC and differential, urinalysis and c-reactive protein, Igs, cytokines and ESR)

In addition, patients are provided with diaries to record for global assessment. Study drug are dispensed.

5.2 PLACEBO RUN-IN PERIOD

During the two-week placebo run-in period, patients are scheduled for two office visits and one additional visit, if necessary. The following assessments are completed at each visit:

Visit 1 (day 7)

body weight

vital signs

ae reporting

baseline joint assessment

grip strength

time required for walking 50 feet

Study drug and patient diaries will be dispensed.

Visit 2 (day 14)

Bodyweight

Vital signs

Joint assessment

Grip strength

Time required for walking 50 feet

General and ra specific laboratory tests

Patient and investigator global assessment

Ae reporting

Study drug and patient diaries will be dispensed.

Patients who meet the eligibility criteria for the study at the end of the two-week placebo run-in period will be randomized into one of four treatment groups.

5.3 DOUBLE-BLIND TREATMENT PERIOD

During the double-blind treatment period, the following procedures will be performed at the specified visits:

Visit 3 (day 7 following randomization)

Body weight

Vital signs

Joint assessment

Grip strength

Time required for walking 50 feet

Global assessment

Ae reporting

Study drug and patient diaries will be dispensed.

Visit 4 (day 14 following randomization)

Body weight

Vital signs

Joint assessment

Grip strength

Time required for walking 50 feet

General and ra specific laboratory tests

Global assessment

Ae reporting

Study drug and patient diaries will be dispensed.

Visit 5 (day 21 following randomization)

Body weight

Vital signs

Joint assessment

Grip strength

Time required for walking 50 feet

Global assessment

Ae reporting

Study drug and patient diaries will be dispensed.

Visit 6 (day 28 following randomization)

Physical examination

Body weight

Vital signs

Joint assessment

Grip strength

Time required for walking 50 feet

General and ra specific laboratory tests

Global assessment

Ae reporting

Study drug and patient diaries will be dispensed

Visit 7 (day 35 following randomization)

Body weight

Vital signs

Joint assessment

Grip strength

Time required for walking 50 feet

Global assessment

AE reporting

Study drug and patient diaries will be dispensed.

Visit 8 (day 42 following randomization)

Physical examination

Body weight

Vital signs

Joint assessment

Grip strength

Time required for walking 50 feet

General and ra specific laboratory tests

Global assessment

AE reporting

Completed study termination report

Visit 9 (at time of termination from the study, if earlier than visit 8)

Physical examination

Body weight

Vital signs

Joint assessment

Grip strength

Time required for walking 50 feet

General and ra specific laboratory tests

Global assessment

AE reporting

Complete study termination report

TABLE 32
Schedule of activities

| | placebo run-in period | | | | Double-blind treatment period | | | | |
|---|---|---|---|---|---|---|---|---|---|
| | | | Visit | | Visit | Visit | Visit | | |
| | | | 2 | Visit | 4 | 5 | 6 | | Visit 8/9 |
| | Visit 0 | Visit 1 | Day | 3 | Day | Day | Day | Visit 7 | Discontinuation |
| Procedure | Screening | Day -7 | 14 | Day 7 | 14 | 21 | 28 | Day 35 | Day 42 |
| Randomization | | | X^{A} | | | | | | |
| Informed consent | X | | | | | | | | |
| Medical history | X | | | | | | | | |
| Physical examination | X | | | | | | X | | X |
| Body weight | X | X | X | X | X | X | X | X | X |
| Height | X | | | | | | | | |
| Vital signs | X | X | X | X | X | X | X | X | X |
| Joint assessment | X | X | X | X | X | X | X | X | X |
| Grip strength | X | X | X | X | X | X | X | X | X |
| Time required for 50 | X | X | X | X | X | X | X | X | X |
| feet | | | | | | | | | |
| Ra specific lab tests | | | X | | X | | X | | X |
| General laboratory tests | X | | X | | X | | X | | X |
| Adverse event reporting | | X | X | X | X | X | X | X | X |
| Dispense study drug | X | | X | X | X | X | X | X | |
| Dispense diary | X | | X | X | X | X | X | X | |
| Patient global | X | | X | X | X | X | X | X | X |
| assessment | | | | | | | | | |
| Investigator global | X | | X | X | X | X | X | X | X |
| assessment | | | | | | | | | |

^{A}patients who meet eligibility criteria at visit 2 will be randomized at visit 2;

6. Patient Discontinuation

If a patient withdraws from the study due to any reason, all visit 9 procedures are to be performed.

7. Statistical methods

7.1 Overview

This study is designed to evaluate the safety and efficacy of three doses of aht-323 (low, medium, and high) on patients with RA.

The primary objective of this study is to evaluate the dose-related change in joint tenderness/pain from baseline on patients with RA.

The secondary objective of this study is to evaluate the dose-related change in assessment of joint swelling from baseline on patients with RA.

The other objectives of this study are to evaluate the following parameters:

Adverse events;

Vital signs throughout the study;

General laboratory measurements;

Grip strength;

Time required to walk 50 feet;

Measurement of the erythrocyte sedimentation rate;

Measurement of the c-reactive protein level;

Improvement assessment;

Patient and investigator global assessments.

7.2 Test of hypotheses

The analysis of the primary efficacy variable will be based on the test of the null hypothesis that there is no treatment effect on the dose-related changes in joint tenderness/pain from baseline, while the alternative hypothesis is that the response rate of patients in the study drug or higher dose group is 25% greater than that of the placebo or lower dose group, respectively.

The analysis of the secondary efficacy variable will be based on the test of the null hypothesis that there is no treatment effect on the dose-related changes in swelling from baseline, while the alternative hypothesis is that the response rate of patients in the study drug or higher dose group is 25% greater than that of the placebo or lower dose group, respectively.

The analysis of the improvement efficacy variable will be based on the test of the null hypothesis that there is no treatment effect on the dose-related changes in improvement from baseline, while the alternative hypothesis is that the improvement of patients in the study drug or higher dose group is 25% greater than that of the placebo or lower dose group, respectively.

Other relevant hypothesis tests may be performed on variables of interest to test the treatment effect.

7.3 Statistical significant level

A 25% change in the aht-323 group compared with placebo is considered clinically significant. A response rate of patients in the placebo group is assumed to be 30%. A two-sided test at a significance level of 0.05 for efficacy, and 0.10 for safety will be applied. The probability of committing type ii error is 20%, i.e., the power is 80%.

7.4 Sample size consideration

A total sample size of 224 patients (56 patients at each of the four treatment groups) are required for detection of a 25% difference (chow & liu, 1998). Approximately 280 patients (70 patients per group) will be enrolled to allow for a 20% dropout.

7.5 Demographics and baseline information

Pre-randomization age, gender, height, weigh, vital signs, and other baseline characteristics will be summarized. Baseline compatibility will be examined.

7.6. Efficacy evaluation

The data will be analyzed using an intent-to-treat approach. Consistent with this approach, all randomized patients who have at least one post-baseline value will be included in the efficacy analysis. A post-baseline value is defined as the result of an evaluation of the patient after the first dose of drug post randomization has been taken.

Continuous variables will be analyzed using an analysis of covariance model, with fixed effects for the treatment groups and the baseline measurement as the covariant; relevant interactions may also be included in the model. Qualitative variables will be presented as incidence rates (n, number, and percent). Variables will be compared between placebo and each dose group using appropriate methods as the following:

Numbers of patients withdrawing due to treatment failure were compared between the aht-323 and placebo groups by fisher's exact test.

Results of all arthritis assessments will be analyzed by comparing least-squares mean changes from baseline between the study drug and placebo groups, using analysis of covariance.

A further composite measure of efficacy in RA, modified from the American college of Rheumatology (ACR) criteria, will be used to assess improvement. In this measure, patients will be classified as improved if they experienced at least a 25% improvement

(a) The number of tender/painful joints,

(b) The number of swollen joints, and from baseline in

(c) At least 3 of the following 4 assessments: physician's global assessment, patient's global assessment, patient's assessment on arthritis pain, and c-reactive protein concentration.

Percentages of patients showing improvement in this measure will be compared using the Cochran-Mantel-Haenszel test.

7.6 Safety assessment

All randomized patients who receive at least one dose of study drug will be included in the safety analysis.

Generally, safety variables will be summarized for each treatment group.

Adverse events will be presented and summarized for each treatment group. Fisher's exact test will be performed to test whether there is an association between adverse events and the treatment groups.

The frequency and timing of patient discontinuations and reasons will be summarized.

The actual value and change from baseline for weight, systolic and diastolic blood pressure, and heart rate will be summarized using descriptive statistics for each treatment group at each visit.

Laboratory results will be compared between placebo and each dose group in terms of:

1) Frequency tables of ‘below lower limit of normal’/‘within normal range’/‘above upper limit of normal’ at relevant visits and shift tables, and

2) Central tendency of the actual values and change from baseline over time.

Mcnemar and stewart-maxwell tests will be performed to analyze the laboratory results.

Physical examination results will be summarized for each treatment group, and comparison will be made to identify any changes from visit 0.

8. Termination of the study

Conditions that may warrant study termination include, but are not limited to, the discovery of a significant, unexpected, and unacceptable risk to the patients, failure of the investigator to enroll patients at an acceptable rate, insufficient adherence to the protocol requirements, completion of study objectives, or at the discretion of the sponsor.

The following references provide further explanation of the above example and are incorporated by reference in their entirety:

1. Simor L S, Lanza F L, Lipsky P E, Hubbard R C, Talwalker S, Schwartz B D, Isakson P C, Geis G S. Preliminary Study Of The Safety And Efficacy Of SC-58635, A Novel Cyclooxygenase 2 Inhibitor: Efficacy And Safety In Two Placebo-Controlled Trials In Osteoarthritis And Rheumatoid Arthritis, And Studies Of Gastrointestinal And Platelet Effects. Arthritis & Rheumatism: Vol.41, No.9, September 1998, pp 1591-1602

2. The Cooperating Clinics Committee of the American Rheumatism Association. A Seven-Day Variability Study of 499 Patients with Peripheral Rheumatoid Arthritis. Arthritis & Rheumatism: Vol.8, No.2, April 1965, pp 302-334

3. Ritchie D M, Boyle J A, mcinnes J M, Jasani M K, Dalakos T G, Grieveson P, et al. Clinical Studies with an Arthcular Index for the Assessment of Joint Tenderness in Patients with Rheumatoid Arthritis. QJM: Vol. 37, 1968, pp 393-406

4. Felson D T, Anderson J J, Boers M, Bombardier C, Furst D, Goldsmith C, et al. American College of Rheumatology Preliminary Definition Of Improvement In Rheumatoid Arthritis. Arthritis & Rheumatism: Vol. 38, 1995, pp 727-735

5. Williams H. J, Willkens R. F,Samuelson Jr C. O., et al. Comparison of Low-dose Oral Pulse Methotrexate and Placebo in the Treatment of Rheumatoid Arthritis. Arthritis & Rheumatism: Vol. 28, No.7, July 1985, pp 721-729

6. Chow S. C. Liu J. P. Design and Analysis of Clinical Trials. John Wiley & Sons, 1998, Chapter 10, New York, N.Y.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

I claim:

1. A method of suppressing the immune system in a patient suffering from an autoimmune disease, comprising daily administering an effective treatment amount of Herba epimedium Extract, an effective treatment amount of Rhizoma Drynaria fortunei (kze.) J. Sm Extract and an effective treatment amount of Radix Tripterygium hypoglaucum (L'evl.) Hutch Extract to said patient, wherein said Herba epimedium Extract contains about 0.035% to about 0.045% by weight of triptolide (C₂₀H₂₄O₆), said Rhizoma Drynaria fortunei (kze.) J. Sm Extract contains about 40% to about 50% by weight of naringin (C₂₇H₃₂O₁₄.2H₂O), and said Radix Tripterygium hypoglaucum (L'evl.) Hutch Extract contains about 10% to about 20% of icariin (C₃₃H₄₀O₁₅)

2. The method of claim 1, wherein about 60 to about 360 mg of Herba epimedium Extract, about 25 to about 150 mg of Rhizoma Drynaria fortunei (kze.) J. Sm Extract and about 25 to about 150 mg of Radix Tripterygium hypoglaucum (L'evl.) Hutch Extract are administered to said patient.

3. The method of claim 1, wherein said autoimmune disease is rheumatoid arthritis.

4. A method of suppressing the immune system in a patient suffering from an autoimmune disease, comprising administering an effective treatment amount of AHT-323 to said patient.

5. The method of claim 1, wherein said patient is administered with AHT-323 at a daily dosage of about 200 mg to 1,200 mg.

6. The method of claim 4, wherein said autoimmune disease is rheumatoid arthritis.

7. A method of inhibiting inflammation in a patient suffering from an inflammatory disorder, comprising daily administering an effective treatment amount of Herba epimedium Extract, an effective treatment amount of Rhizoma Drynaria fortunei (kze.) J. Sm Extract and an effective treatment amount of Radix Tripterygium hypoglaucum (L'evl.) Hutch Extract to said patient, wherein said Herba epimedium Extract contains about 0.035% to about 0.045% by weight of triptolide (C₂₀H₂₄O₆), said Rhizoma Drynaria fortunei (kze.) J. Sm Extract contains about 40% to about 50% by weight of naringin (C₂₇H₃₂O₁₄.2H₂O), and said Radix Tripterygium hypoglaucum (L'evl.) Hutch Extract contains about 10% to about 20% of icariin (C₃₃H₄₀O_{15).}

8. The method of claim 7, wherein about 60 to about 360 mg of Herba epimedium Extract, about 25 to about 150 mg of Rhizoma Drynaria fortunei (kze.) J. Sm Extract and about 25 to about 150 mg of Radix Tripterygium hypoglaucum (L'evl.) Hutch Extract are administered to said patient.

9. The method of claim 7, wherein said inflammatory disorder results from rheumatoid arthritis.

10. A method of inhibiting inflammation in a patient suffering from an inflammatory disorder, comprising administering an effective treatment amount of AHT-323 to said patient.

11. The method of claim 10, wherein said patient is administered with AHT-323 at a daily dosage of about 200 mg to 1,200 mg.

12. The method of claim 11, wherein said inflammatory disorder results from rheumatoid arthritis.

13. A method of relieving pain in a patient subject to said pain, comprising daily administering an effective treatment amount of Herba epimedium Extract, an effective treatment amount of Rhizoma Drynaria fortunei okze.) J. Sm Extract and an effective treatment amount of Radix Tripterygium hypoglaucum (L'evl.) Hutch Extract to said patient, wherein said Herba epimedium Extract contains about 0.035% to about 0.045% by weight of triptolide (C₂₀H₂₄O₆), said Rhizoma Drynaria fortunei (kze.) J. Sm Extract contains about 40% to about 50% by weight of naringin (C₂₇H₃₂O₁₄.2H₂O), and said Radix Tripterygium hypoglaucum (L'evl.) Hutch Extract contains about 10% to about 20% of icariin (C₃₃H₄₀O₁₅).

14. The method of claim 13, wherein about 60 to about 360 mg of Herba epimedium Extract, about 25 to about 150 mg of Rhizoma Drynaria fortunei (kze.) J. Sm Extract and about 25 to about 150 mg of Radix Tripterygium hypoglaucum (L'evl.) Hutch Extract are administered to said patient.

15. The method of claim 13, wherein said pain results from an inflammatory disorder.

16. The method of claim 15, wherein said inflammatory disorder results from rheumatoid arthritis.

17. A method of relieving pain in a patient subject to said pain, comprising administering an effective treatment amount of AHT-323 to said patient.

18. The method of claim 17, wherein said patient is administered with AHT-323 to said patient at a daily dosage of about 200 mg to 1,200 mg.

19. The method of claim 17, wherein said pain results from an inflammatory disorder.

20. The method of claim 19, wherein said inflammatory disorder results from rheumatoid arthritis.

21. A method of reducing hypersensitivity in a patient subject to said hypersensitivity, comprising daily administering Herba epimedium Extract, Rhizoma Drynaria fortunei (kze.) J. Sm Extract and Radix Tripterygium hypoglaucum (L'evl.) Hutch Extract to said patient.

22. The method of claim 21, wherein said patient is administered about 60 to about 360 mg of Herba epimedium Extract, about 25 to about 150 mg of Rhizoma Drynaria fortunei (kze.) J. Sm Extract and about 25 to about 150 mg of Radix Tripterygium hypoglaucum (L'evl.) Hutch Extract, said Herba epimedium Extract contains about 0.035% to about 0.045% by weight of triptolide (C₂₀H₂₄O₆), said Rhizoma Drynaria fortunei (kze.) J. Sm Extract contains about 40% to about 50% by weight of naringin (C₂₇H₃₂O₁₄.2H₂O), and said Radix Tripterygium hypoglaucum (L'evl.) Hutch Extract contains about 10% to about 20% of icariin (C₃₃H₄₀O₁₅).

23. A method of reducing hypersensitivity in a patient subject to said hypersensitivity, comprising daily administering AHT-323 to said patient.

24. The method of claim 23, wherein said patient is administered with AHT-323 to said patient at a daily dosage of about 200 mg to 1,200 mg.

25. A Tripterygium hypoglaucum (L'evl.) Hutch extract, comprising about 0.035% to about 0.045% by weight of triptolide.

26. A Herba epimedium extract, comprising about 10% to about 20% by weight of icarimn.

27. A Drynaria fortunei (kze.) J. Sm. Extract, comprising about 40% to about 50% by weight of naringin.

28. A therapeutic herbal composition, comprising a Tripterygium hypoglaucum (L'evl.) Hutch extract, which contains about 0.035% to about 0.045% by weight of triptolide, a Herba epimedium extract, which contains about 10% to about 20% by weight of icariin and a Drynaria fortunei (kze.) J. Sm. Extract, which contains about 40% to about 50% by weight of naringin.

29. The composition of claim 28, wherein said composition comprises 0.5-1.5 parts of said Tripterygium hypoglaucum (L'evl.) Hutch extract, 2-3 parts of said Herba epimedium extract and 0.5-1.5 parts of said Drynaria fortunei (kze.) J. Sm. Extract, by weight.

30. The composition of claim 29, wherein said composition comprises one part of said Tripterygium hypoglaucum (L'evl.) Hutch extract, 2.4 parts of said Herba epimedium extract and one part of said Drynaria fortunei kze.) J. Sm. Extract, by weight.

31. The composition of claim 28, further comprising dextrin.

32. The composition of claim 31, further comprising 3.0-4.0 parts of dextrin.

33. The composition of claim 32, further comprising 3.6 parts of dextrin.

34. A method of preparing Tripterygium hypoglaucum (L'evl.) Hutch extract having triptolide in a range from about 0.035% to about 0.045% by weight, comprising the steps of
a. preparing Tripterygium hypoglaucum (L'evl.) Hutch roots as a raw material;
b. extracting said raw material with boiling water;
c. applying said boiling water extract to a chromatography column;
d. eluting said column with 70-80% ethanol to produce an ethanol elute;
e. removing said ethanol from said elute such that said elute is concentrated;
f. drying said concentrated elute until it becomes powder; and
g. passing said powder through a particle sieve and collecting said passing through powder.

35. The method of claim 34, further comprising the step of soaking said raw material in water for about at least two hours before extracting said plant with boiling water.

36. The method of claim 35., wherein said raw material is soaked in water for about 12 hours.

37. The method of claim 34, further comprising the step of filtering said boiling water extract through a filter.

38. The method of claim 34., wherein said raw material is extract in boiling water for about one to five hours.

39. The method of claim 38, wherein said raw material is extract in boiling water for about two hours.

40. The method of claim 34, wherein said chromatography column is made of big-pore polymeric absorbent.

41. The method of claim 34, wherein said particle sieve has size 80-mesh.

42. A method of preparing Tripterygium hypoglaucum (L'evl.) Hutch extract having triptolide in a range from about 0.035% to about 0.045% by weight, comprising the steps of
a.preparing Tripterygium hypoglaucum (L'evl.) Hutch roots as a raw material;
b.soaking said raw material with water for at least two hours;
c.extracting said raw material with boiling water for about one to five hours after step b;
d.applying said boiling water extract to a chromatography column made of big-pore polymeric aborbent;
e.eluting said column with 70-80% ethanol to produce an ethanol elute;
f.removing said ethanol from said elute such that said elute is concentrated;
g.drying said concentrated elute until it becomes powder; and
h. passing said powder through a 80-mesh particle sieve and collecting said passing through powder.

43. A method of preparing a Drynaria fortunei (Kze.) J. Sm. extract having narignin in a range from about 40% to about 50% by weight, comprising the steps of
a. preparing Drynaria fortunei (Kze.) J. Sm. rhizome as a raw material;
b. grinding said raw material to produce a powder;
c. soaking said powder in 95% ethanol at an elevated temperature to produce an ethanol extract absent insoluble materials;
d. adding hot water to said ethanol extract to produce an ethanol-water solution and let said solution stay for at least two hours;
e. removing ethanol from said solution so as to concentrate said solution;
f. diluting said concentrated solution with water;
g. applying said diluted solution to a chromatography column;
h. eluting said column with 80% ethanol to produce an ethanol elute;
i. removing ethanol from said ethanol elute so as to concentrate said elute;
j. drying said concentrated elute until it becomes a powder.

44. The method of claim 43, wherein said raw material powder is soaked in 95% ethanol at a volume five times of the weight of said raw material powder.

45. The method of claim 44, wherein said raw material powder is soaked in 95% ethanol for at least eight hours at 60° C.

46. The method of claim 45, wherein said raw material powder is soaked in 95% ethanol for about 16 hours at 60° C.

47. The method of claim 43, wherein said hot water is added to said 95% ethanol extract at a ratio of about one part of said water to about 10 parts of said ethanol extract.

48. The method of claim 43, wherein said ethanol-water solution stays overnight.

49. The method of claim 43, wherein said concentrated solution is diluted with about two volumes of water prior to the application to said column.

50. The method of claim 43, wherein said chromatography column is a D101 big-pore polymeric absorbent chromatography column.

51. The method of claim 43, further comprising the step of washing said chromatography column with 20% ethanol prior to said elution.

52. A method of preparing a Drynaria fortunei (Kze.) J. Sm. extract having narignin in a range from about 40% to about 50% by weight, comprising the steps of
a. preparing Drynaria fortunei (Kze.) J. Sm. rhizome as a raw material;
b. grinding said raw material to produce a powder;
c. soaking said powder in 95% ethanol at a volume five times of said powder by weight for about 16 hours at a 60° C. to produce an ethanol extract absent insoluble materials;
d. adding about 1/10 volumes of hot water to said ethanol extract to produce an ethanol-water solution and let said solution stay overnight;
e. removing ethanol from said solution so as to concentrate said solution;
f. diluting said concentrated solution with two volumes of water;
g. applying said diluted solution to a D101 big-pore polymeric absorbent chromatography column;
h. washing said loaded chromatography column with 20% ethanol;
i. eluting said column with 80% ethanol to produce an ethanol elute;
j. removing ethanol from said ethanol elute so as to concentrate said elute;
k. drying said concentrated elute until it becomes a powder.

53. A method of preparing a Herba epimedium extract having icariin content in a range from about 10% to about 20% by weight, comprising the steps of
a. preparing Herba epimedium leaves as a raw material;
b. grinding said raw material to become a powder;
C. boiling said powder in water to produce a boiling water extract absent insoluble materials;
d. applying said boiling water extract to a chromatography column;
e. eluting said column with 50% ethanol to produce an ethanol elute;
f. removing ethanol from said ethanol elute so as to concentrate said ethanol elute; and
h. drying said concentrated ethanol elute until it becomes a powder.

54. The method of claim 53, wherein said raw material powder is boiled in 10 volumes of water (v/w).

55. The method of claim 53, wherein said raw material powder is boiled in water for about 30 minutes to 2.5 hours.

56. The method of claim 55, wherein said raw material powder is boiled in water for about 1.5 hours.

57. The method of claim 53, wherein said chromatography column is a polyamide column;

58. The method of claim 57, wherein said polyamide column has a pore size in a range of 30-60 mesh.

59. The method of claim 57, wherein said polyamide column has polyamide in an amount twice as much as said raw material powder by weight.

60. The method of claim 57, further comprising the step of washing said loaded chromatography column with water until the water elute becomes colorless.

61. A method of preparing a Herba epimedium extract having icariin content in a range from about 10% to about 20% by weight, comprising the steps of
a. preparing Herba epimedium leaves as a raw material;
b. grinding said raw material to become a powder;
c. boiling said powder in 10 volumes of water (v/w) for about 30 minutes to about 2.5 hours to produce a boiling water extract absent insoluble materials;
d. applying said boiling water extract to a 30-60 mesh polyamide chromatography column, said column has polyamide in an amount twice as much as said raw material powder by weight;
e. washing said loaded chromatography column with water until the water elute becomes colorless;
f. eluting said column with 50% ethanol to produce an ethanol elute;
g. removing ethanol from said ethanol elute so as to concentrate said ethanol elute; and
h. drying said concentrated ethanol elute until it becomes a powder.
